SEQUENCE LISTING (38 sequences)

SEQ ID NO: 1 - DNA, 10186 bp, Artificial Sequence
  Synthetic Modified Lentiviral Transfer Plasmid (complete)
tccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggttcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagccctcgagccaccatgcagctggaactgtctacctgtgtgtttctgtgtctgctgcctctggggttttctgctatcaggagatactatctgggagctgtggagctgtcctgggactacaggcagtctgagctgctgagagaactgcatgtggataccagattcccagctacagctccaggagctctgcctctgggcccatctgtgctgtacaagaaaacagtctttgtggagtttacagaccagctgttctctgtggccaggccaagaccaccttggatgggactgctgggaccaaccatccaggctgaggtgtatgatacagtggtggtgaccctgaaaaacatggcctcccatcctgtgagcctgcatgctgtgggggtgtccttctggaagtcctctgagggagctgagtatgaagaccatacctcccagagggagaaagaagatgataaggtgctgcctggcaaaagccagacctatgtctggcaggtgctgaaggagaatggaccaactgcttctgacccaccatgcctgacctactcttatctgtcccatgtggatctggtgaaggacctgaattctggactgattggagctctgctggtgtgtagagagggaagcctgaccagagaaagaacccagaacctgcatgagtttgtcctgctgtttgctgtgtttgatgaagggaagagctggcactctgccaggaatgactcctggaccagagctatggatccagctcctgctagagctcagcctgctatgcacacagtcaatggctatgtgaataggtctctgccaggactgattggctgccataagaaatctgtctattggcatgtgattggaatgggcaccagccctgaggtgcattctatcttcctggaaggccacacctttctggtcaggcaccatagacaggcctctctggagatctcccctctgaccttcctgacagctcagacctttctgatggacctggggcagttcctgctgttttgccatatctcttcccaccatcatggaggaatggaggctcatgtcagggtggaatcctgtgctgaggaaccacagctgagaagaaaggctgatgaggaagaggactatgatgataacctgtatgactctgatatggatgtggtgaggctggatggggatgatgtcagccctttcatccagatcaggtctgtggccaagaaacatccaaagacctgggtccactacattgctgctgaagaggaagattgggactatgcccccctggtgctggctcctgatgatagatcctacaaaagccagtatctgaacaatgggccccagaggattggaaggaagtacaagaaagtgaggttcatggcctatacagatgagacctttaagaccagagaggctatccagcatgaatctgggatcctgggacctctgctgtatggagaagtgggggataccctgctgatcatcttcaagaaccaggcctccaggccatacaatatctatccccatggcatcacagatgtgagaccactgtacagcaggagactgcccaagggggtcaaacacctgaaggatttccccatcctgcctggagagatctttaagtataaatggacagtcacagtggaagatgggcctaccaagtctgatccaaggtgcctgaccagatactatagctcttttgtgaacatggagagagacctggcttctggactgattggacccctgctgatctgttacaaagagtctgtggaccagaggggcaaccagatcatgtctgataagagaaatgtcatcctgttctctgtgtttgatgagaacaggagctggtacctgacagagaacatccagaggttcctgccaaatccagctggagtgcagctggaggacccagaatttcaggcttccaacatcatgcatagcatcaatggctatgtgtttgatagcctgcagctgtctgtctgcctgcatgaggtggcctactggtatatcctgtccattggagctcagacagacttcctgtctgtgttctttagtgggtacacctttaagcataaaatggtgtatgaggataccctgaccctgttccccttttctggggagacagtgttcatgtccatggaaaaccctggcctgtggatcctggggtgccacaactctgacttcaggaatagaggaatgacagccctgctgaaagtgtccagctgtgataagaatacaggggattactatgaggactcttatgaagatatctctgcttatctgctgagcaagaacaatgccattgagcccaggtcttttgctcagaactccagacctccatctgcttctgctcctaagccacctgtgctgagaagacatcagagggacatctccctgcctaccttccagccagaggaagataaaatggactatgatgatatcttcagcacagagaccaagggggaagattttgacatctatggagaggatgaaaaccaggatccaagatccttccagaagagaaccagacactactttattgctgctgtggagcagctgtgggactatgggatgtctgaaagcccaagggccctgaggaacagagctcagaatggagaggtgcccagattcaagaaagtggtgttcagagagtttgctgatggcagctttacccagccatcttacaggggggagctgaacaagcatctggggctgctgggaccctatatcagagctgaggtggaagataacatcatggtgaccttcaagaatcaggcttctaggccctactccttttattcttccctgatctcctaccctgatgatcaggagcagggagctgaacctaggcacaactttgtgcagccaaatgagaccagaacctacttttggaaggtgcagcatcacatggctcccacagaggatgaatttgactgcaaagcttgggcctatttttctgatgtggacctggagaaggatgtgcattctggcctgattgggcctctgctgatctgtagggccaacaccctgaatgctgctcatggaagacaggtcacagtgcaggagtttgctctgttctttaccatctttgatgaaaccaagagctggtacttcacagagaatgtggaaaggaattgcagagccccctgtcatctgcagatggaggaccctaccctgaaggaaaactacaggttccatgccatcaatggatatgtcatggataccctgcctggcctggtcatggctcagaaccagaggatcagatggtacctgctgtctatgggatccaatgagaatatccatagcatccacttctctggccatgtcttttctgtgaggaagaaagaggaatacaaaatggctgtgtacaatctgtatcctggggtctttgagacagtggaaatgctgccaagcaaagtgggaatctggagaattgagtgcctgattggggaacacctgcaggctgggatgagcaccaccttcctggtgtactctaagaaatgtcagaccccactggggatggcctctggacatatcagggacttccagatcacagcttctggacagtatggacagtgggctccaaagctggctagactgcactattctggctccatcaatgcctggtctaccaaagagccattctcctggatcaaggtggacctgctggcccccatgatcatccatggaatcaaaacccagggagctaggcagaagttcagctctctgtacatctcccagtttatcatcatgtatagcctggatgggaagaaatggcagacctacagaggcaattccactgggaccctgatggtcttctttggaaatgtggattcctctggcatcaagcacaacatcttcaatccacccatcattgccaggtacatcaggctgcatcctacccactatagcatcaggtctaccctgagaatggagctgatgggatgtgacctgaacagctgttctatgccactgggcatggagtccaaggctatctctgatgcccagatcacagcttcttcctacttcaccaatatgtttgctacctggtccccaagcaaggctagactgcacctgcagggaagatccaatgcttggagaccccaggtgaacaatcctaaggagtggctgcaggtggacttccagaaaaccatgaaggtcacaggggtgaccacccagggagtgaaatctctgctgacctccatgtatgtcaaggagttcctgatcagctcttcccaggatggccaccagtggaccctgttctttcagaatggcaaggtcaaagtgttccaggggaatcaggactcttttaccccagtggtgaactccctggatcctccactgctgaccaggtacctgagaatccatcctcagagctgggtgcaccagattgctctgagaatggaggtcctgggatgtgaagctcaggacctgtattgagcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgcctta

SEQ ID NO: 2 - DNA, 659 bp, Artificial Sequence
  Synthetic CD68 promoter
tcatttcttacctccccttccctctcccacctgctactgggtgcatctctgctccccccttccccagcagatggttacctttgggctgttgctttcttgtcaccatctgagttctcagacgctggaaagccatgttctcggctctgtgaatgacaatgctgactggagtgctgcccctctgtaaagggctgggtgtggatggtcacaagcccctcacatgcctcagccaagaggaagtagtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcc

SEQ ID NO: 3 - DNA, 4404 bp, Artificial Sequence
  Synthetic ET3 LCO
atgcagctggaactgtctacctgtgtgtttctgtgtctgctgcctctggggttttctgctatcaggagatactatctgggagctgtggagctgtcctgggactacaggcagtctgagctgctgagagaactgcatgtggataccagattcccagctacagctccaggagctctgcctctgggcccatctgtgctgtacaagaaaacagtctttgtggagtttacagaccagctgttctctgtggccaggccaagaccaccttggatgggactgctgggaccaaccatccaggctgaggtgtatgatacagtggtggtgaccctgaaaaacatggcctcccatcctgtgagcctgcatgctgtgggggtgtccttctggaagtcctctgagggagctgagtatgaagaccatacctcccagagggagaaagaagatgataaggtgctgcctggcaaaagccagacctatgtctggcaggtgctgaaggagaatggaccaactgcttctgacccaccatgcctgacctactcttatctgtcccatgtggatctggtgaaggacctgaattctggactgattggagctctgctggtgtgtagagagggaagcctgaccagagaaagaacccagaacctgcatgagtttgtcctgctgtttgctgtgtttgatgaagggaagagctggcactctgccaggaatgactcctggaccagagctatggatccagctcctgctagagctcagcctgctatgcacacagtcaatggctatgtgaataggtctctgccaggactgattggctgccataagaaatctgtctattggcatgtgattggaatgggcaccagccctgaggtgcattctatcttcctggaaggccacacctttctggtcaggcaccatagacaggcctctctggagatctcccctctgaccttcctgacagctcagacctttctgatggacctggggcagttcctgctgttttgccatatctcttcccaccatcatggaggaatggaggctcatgtcagggtggaatcctgtgctgaggaaccacagctgagaagaaaggctgatgaggaagaggactatgatgataacctgtatgactctgatatggatgtggtgaggctggatggggatgatgtcagccctttcatccagatcaggtctgtggccaagaaacatccaaagacctgggtccactacattgctgctgaagaggaagattgggactatgcccccctggtgctggctcctgatgatagatcctacaaaagccagtatctgaacaatgggccccagaggattggaaggaagtacaagaaagtgaggttcatggcctatacagatgagacctttaagaccagagaggctatccagcatgaatctgggatcctgggacctctgctgtatggagaagtgggggataccctgctgatcatcttcaagaaccaggcctccaggccatacaatatctatccccatggcatcacagatgtgagaccactgtacagcaggagactgcccaagggggtcaaacacctgaaggatttccccatcctgcctggagagatctttaagtataaatggacagtcacagtggaagatgggcctaccaagtctgatccaaggtgcctgaccagatactatagctcttttgtgaacatggagagagacctggcttctggactgattggacccctgctgatctgttacaaagagtctgtggaccagaggggcaaccagatcatgtctgataagagaaatgtcatcctgttctctgtgtttgatgagaacaggagctggtacctgacagagaacatccagaggttcctgccaaatccagctggagtgcagctggaggacccagaatttcaggcttccaacatcatgcatagcatcaatggctatgtgtttgatagcctgcagctgtctgtctgcctgcatgaggtggcctactggtatatcctgtccattggagctcagacagacttcctgtctgtgttctttagtgggtacacctttaagcataaaatggtgtatgaggataccctgaccctgttccccttttctggggagacagtgttcatgtccatggaaaaccctggcctgtggatcctggggtgccacaactctgacttcaggaatagaggaatgacagccctgctgaaagtgtccagctgtgataagaatacaggggattactatgaggactcttatgaagatatctctgcttatctgctgagcaagaacaatgccattgagcccaggtcttttgctcagaactccagacctccatctgcttctgctcctaagccacctgtgctgagaagacatcagagggacatctccctgcctaccttccagccagaggaagataaaatggactatgatgatatcttcagcacagagaccaagggggaagattttgacatctatggagaggatgaaaaccaggatccaagatccttccagaagagaaccagacactactttattgctgctgtggagcagctgtgggactatgggatgtctgaaagcccaagggccctgaggaacagagctcagaatggagaggtgcccagattcaagaaagtggtgttcagagagtttgctgatggcagctttacccagccatcttacaggggggagctgaacaagcatctggggctgctgggaccctatatcagagctgaggtggaagataacatcatggtgaccttcaagaatcaggcttctaggccctactccttttattcttccctgatctcctaccctgatgatcaggagcagggagctgaacctaggcacaactttgtgcagccaaatgagaccagaacctacttttggaaggtgcagcatcacatggctcccacagaggatgaatttgactgcaaagcttgggcctatttttctgatgtggacctggagaaggatgtgcattctggcctgattgggcctctgctgatctgtagggccaacaccctgaatgctgctcatggaagacaggtcacagtgcaggagtttgctctgttctttaccatctttgatgaaaccaagagctggtacttcacagagaatgtggaaaggaattgcagagccccctgtcatctgcagatggaggaccctaccctgaaggaaaactacaggttccatgccatcaatggatatgtcatggataccctgcctggcctggtcatggctcagaaccagaggatcagatggtacctgctgtctatgggatccaatgagaatatccatagcatccacttctctggccatgtcttttctgtgaggaagaaagaggaatacaaaatggctgtgtacaatctgtatcctggggtctttgagacagtggaaatgctgccaagcaaagtgggaatctggagaattgagtgcctgattggggaacacctgcaggctgggatgagcaccaccttcctggtgtactctaagaaatgtcagaccccactggggatggcctctggacatatcagggacttccagatcacagcttctggacagtatggacagtgggctccaaagctggctagactgcactattctggctccatcaatgcctggtctaccaaagagccattctcctggatcaaggtggacctgctggcccccatgatcatccatggaatcaaaacccagggagctaggcagaagttcagctctctgtacatctcccagtttatcatcatgtatagcctggatgggaagaaatggcagacctacagaggcaattccactgggaccctgatggtcttctttggaaatgtggattcctctggcatcaagcacaacatcttcaatccacccatcattgccaggtacatcaggctgcatcctacccactatagcatcaggtctaccctgagaatggagctgatgggatgtgacctgaacagctgttctatgccactgggcatggagtccaaggctatctctgatgcccagatcacagcttcttcctacttcaccaatatgtttgctacctggtccccaagcaaggctagactgcacctgcagggaagatccaatgcttggagaccccaggtgaacaatcctaaggagtggctgcaggtggacttccagaaaaccatgaaggtcacaggggtgaccacccagggagtgaaatctctgctgacctccatgtatgtcaaggagttcctgatcagctcttcccaggatggccaccagtggaccctgttctttcagaatggcaaggtcaaagtgttccaggggaatcaggactcttttaccccagtggtgaactccctggatcctccactgctgaccaggtacctgagaatccatcctcagagctgggtgcaccagattgctctgagaatggaggtcctgggatgtgaagctcaggacctgtattga

SEQ ID NO: 4 - protein, 1467 aa, Artificial Sequence
  Synthetic ET3 LCO Amino Acid Seq, synthetic fVIII
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - DNA, 8775 bp, Artificial Sequence
  Synthetic Complete Sequence of plasmid
tcagaaaaactcgtcgagcatcagatgaaactgcaacttattcatatcggggttatcaattccatatttttggaaaagcctcttctgtaaactaggagaaaactcgccgaggcagttccataggatggccagatcctggtatctgtctgcgattccaacccttcccacatcaatacaacctataagtttgccctcgtcaaaaatcaggttatcaagagagaaatcaccgtgagtgacgactgagtctggactgaatggcaaaagtttgtgcatttctttccagacctgttccacgggccagccatttctctcgtcatcgaaatcactagcatccaccaaaccgttattcattcgtgactgggcctgagcgagacgaaatacgcgatcgctgttaaaagggcaattacaaacaggaatggaatgcaaccgtctcaggaacactgccagagcgtcaacaatattttcgcctgaatcagggtattcttccagtacctggaatgctgtctttccggggatcgcagtggtgagtaaccatgcgtcatctggagtacggataaaatgcttgatggttggaagaggcataaattcggtcagccagtttagtctcaccatctcatctgtcacatcattggcaacgctaccttttccatgcttcaggaacagctctggagcatcgggcttcccatacaatcggtagattgtggctccagattgcccgacattgtcgcgagcccatttatacccatataagtcagcgtccatgttggaattgagtctcggcctggacgtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacatatggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcttatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccccatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattaagtagttgattaactaattaggtgattcacctaattagttaatcaactacgtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttgcggccgcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagtttaaacggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 6 - DNA, 47 bp, Artificial Sequence
  Synthetic Synthetic nucleotide sequence, 6 frame stop element
tagttgattaactaattaggtgattcacctaattagttaatcaacta

SEQ ID NO: 7 - DNA, 810 bp, Artificial Sequence
  Synthetic hCD8 codon optimized Kanamycin resistance
tcagaaaaactcgtcgagcatcagatgaaactgcaacttattcatatcggggttatcaattccatatttttggaaaagcctcttctgtaaactaggagaaaactcgccgaggcagttccataggatggccagatcctggtatctgtctgcgattccaacccttcccacatcaatacaacctataagtttgccctcgtcaaaaatcaggttatcaagagagaaatcaccgtgagtgacgactgagtctggactgaatggcaaaagtttgtgcatttctttccagacctgttccacgggccagccatttctctcgtcatcgaaatcactagcatccaccaaaccgttattcattcgtgactgggcctgagcgagacgaaatacgcgatcgctgttaaaagggcaattacaaacaggaatggaatgcaaccgtctcaggaacactgccagagcgtcaacaatattttcgcctgaatcagggtattcttccagtacctggaatgctgtctttccggggatcgcagtggtgagtaaccatgcgtcatctggagtacggataaaatgcttgatggttggaagaggcataaattcggtcagccagtttagtctcaccatctcatctgtcacatcattggcaacgctaccttttccatgcttcaggaacagctctggagcatcgggcttcccatacaatcggtagattgtggctccagattgcccgacattgtcgcgagcccatttatacccatataagtcagcgtccatgttggaattgagtctcggcctggacgtttcccgttgaatgtggctcat

SEQ ID NO: 8 - protein, 269 aa, Artificial Sequence
  Synthetic hCD8 codon optimized Kanamycin resistance
MSHIQRETSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 9 - DNA, 1242 bp, Artificial Sequence
  Synthetic CMV promoter + beta-globin intron
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcttatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 10 - DNA, 462 bp, Artificial Sequence
  Synthetic PGK polyA signal
ctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaa

SEQ ID NO: 11 - DNA, 1503 bp, Artificial Sequence
  Synthetic Gag
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataa

SEQ ID NO: 12 - protein, 500 aa, Artificial Sequence
  Synthetic Gag
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 13 - DNA, 3012 bp, Artificial Sequence
  Synthetic Pol
ttttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattaa

SEQ ID NO: 14 - protein, 1003 aa, Artificial Sequence
  Synthetic Pol AA
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 15 - DNA, 5531 bp, Artificial Sequence
  Synthetic VSVG Plasmid, Complete Sequence
tcagaaaaactcatcgagcatcagatggaactgcaatttattcatgtcagggttatctatgccgtatttttggaaaagccgcttctgcagtgagggagaaaactcaccgaggcagttccagaggatggcaagatcctggtacctgtcggcgattccgactcgtcccacgtcaatgcagcctatgagtttcccctcgtcgaaaataaggttatccagtgagaaatccccgtgagtcacgaccgaatccggactgaatggcaacagcttgtgcatttccttccacacctgctccacaggccagccatttctctcgtcgtcaaaatcgctggcatcaaccagaccgttgttcattctggattgagcctgagcgagacgaaatacgcggtcgctgttaaatggacagttacacacgggaatagagtgcaatctgcgcaggaacactgccagagcgtcaacaatattttcgccactatcgggatattcttctagtacctggaatgctgtctttccggggatcgcagtggtgagtaaccatgcgtcatcaggggtacggatgaaatgcttgatggtcggcagaggcataaattcggtcagccagtttagtctgaccatctcatctgtcacatcattggcaacgctgccttttccgtgcttcagaaacagctctggggcgtcgggcttcccgtacaacctatagattgtggcgccggactgcccgacattgtcccgagcccatttatacccgtacaggtcagcgtccatgttggaatttaatcgtggcctggacgtctcccgctgaatatggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaacatatggggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgagccaccatgaagtgtttgctgtaccttgcgttcctgtttattggggtgaactgcaagttcactatcgtgtttcctcacaaccaaaaaggcaactggaaaaacgtgccttcaaattatcattattgcccctctagtagcgacctgaactggcataatgacttgattgggaccgctctgcaggtgaagatgccaaagagccacaaggctatccaggctgatggttggatgtgtcatgcctctaaatgggtaactacctgtgatttccgctggtatggaccaaaatatataacacacagcatcaggagtttcacaccgagtgttgaacaatgtaaagaaagtatcgaacagactaagcagggtacctggctgaaccctggttttcccccacagtcatgtgggtatgcaaccgtgacagatgctgaggccgtgattgtgcaggtcacgccacatcatgtgcttgttgatgagtacaccggagagtgggtcgattcacagttcataaatggcaagtgctccaactacatttgcccaaccgtgcacaacagcactacttggcacagtgactacaaagttaagggtttatgtgattccaacctgatctccatggatatcacattcttttctgaagacggcgagctgtcctctctaggcaaagaaggaactgggtttcgaagtaattattttgcctacgaaacaggaggaaaagcctgcaagatgcagtattgtaaacactggggcgtgagactacccagcggtgtttggttcgagatggccgataaggacctgtttgcagcagcaagattcccggaatgccctgagggatccagcatttccgctccatcacagacttctgtcgacgttagcctgatccaggatgtggaacgcattctcgactattcgctttgccaggagacctggtcaaagataagagctggacttcccatctcacccgtcgatttatcttacctggccccaaagaatcccggcaccggaccagcgttcaccataatcaatggcacgctcaaatactttgagacgcgttacattcgggtagacattgcagctcctattctctctcggatggtaggtatgatctctgggactacaacagaacgcgagctctgggacgactgggcaccttacgaggatgtcgagatcggacctaacggagtgctccggacctcttcagggtataagtttcctctctatatgatcggccacgggatgttggacagcgatctgcatctgtcttccaaagcccaggtgtttgaacacccacacattcaagacgccgccagccagctgccagatgatgagtcgcttttctttggtgacacaggcttgagtaagaatcccattgagctggtagaaggatggttttcatcctggaaaagttcaatagcttcctttttcttcattattggcttaattatcggcttgtttttggtcctacgtgttggtattcatctttgtatcaaactcaagcatactaagaagcgacaaatctacacagatatcgagatgaataggctgggcaagtgagcggccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttcctaggggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 16 - DNA, 810 bp, Artificial Sequence
  Synthetic Human NK cell codon optimized kanamycin resistance
tcagaaaaactcatcgagcatcagatggaactgcaatttattcatgtcagggttatctatgccgtatttttggaaaagccgcttctgcagtgagggagaaaactcaccgaggcagttccagaggatggcaagatcctggtacctgtcggcgattccgactcgtcccacgtcaatgcagcctatgagtttcccctcgtcgaaaataaggttatccagtgagaaatccccgtgagtcacgaccgaatccggactgaatggcaacagcttgtgcatttccttccacacctgctccacaggccagccatttctctcgtcgtcaaaatcgctggcatcaaccagaccgttgttcattctggattgagcctgagcgagacgaaatacgcggtcgctgttaaatggacagttacacacgggaatagagtgcaatctgcgcaggaacactgccagagcgtcaacaatattttcgccactatcgggatattcttctagtacctggaatgctgtctttccggggatcgcagtggtgagtaaccatgcgtcatcaggggtacggatgaaatgcttgatggtcggcagaggcataaattcggtcagccagtttagtctgaccatctcatctgtcacatcattggcaacgctgccttttccgtgcttcagaaacagctctggggcgtcgggcttcccgtacaacctatagattgtggcgccggactgcccgacattgtcccgagcccatttatacccgtacaggtcagcgtccatgttggaatttaatcgtggcctggacgtctcccgctgaatatggctcat

SEQ ID NO: 17 - protein, 511 aa, Artificial Sequence
  Synthetic Amino Acid of SEQ ID NO 16
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - DNA, 1267 bp, Artificial Sequence
  Synthetic PGK promoter/PGK intron
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacag

SEQ ID NO: 19 - DNA, 1536 bp, Artificial Sequence
  Synthetic 293T codon optimized VSVG coding sequence
atgaagtgtttgctgtaccttgcgttcctgtttattggggtgaactgcaagttcactatcgtgtttcctcacaaccaaaaaggcaactggaaaaacgtgccttcaaattatcattattgcccctctagtagcgacctgaactggcataatgacttgattgggaccgctctgcaggtgaagatgccaaagagccacaaggctatccaggctgatggttggatgtgtcatgcctctaaatgggtaactacctgtgatttccgctggtatggaccaaaatatataacacacagcatcaggagtttcacaccgagtgttgaacaatgtaaagaaagtatcgaacagactaagcagggtacctggctgaaccctggttttcccccacagtcatgtgggtatgcaaccgtgacagatgctgaggccgtgattgtgcaggtcacgccacatcatgtgcttgttgatgagtacaccggagagtgggtcgattcacagttcataaatggcaagtgctccaactacatttgcccaaccgtgcacaacagcactacttggcacagtgactacaaagttaagggtttatgtgattccaacctgatctccatggatatcacattcttttctgaagacggcgagctgtcctctctaggcaaagaaggaactgggtttcgaagtaattattttgcctacgaaacaggaggaaaagcctgcaagatgcagtattgtaaacactggggcgtgagactacccagcggtgtttggttcgagatggccgataaggacctgtttgcagcagcaagattcccggaatgccctgagggatccagcatttccgctccatcacagacttctgtcgacgttagcctgatccaggatgtggaacgcattctcgactattcgctttgccaggagacctggtcaaagataagagctggacttcccatctcacccgtcgatttatcttacctggccccaaagaatcccggcaccggaccagcgttcaccataatcaatggcacgctcaaatactttgagacgcgttacattcgggtagacattgcagctcctattctctctcggatggtaggtatgatctctgggactacaacagaacgcgagctctgggacgactgggcaccttacgaggatgtcgagatcggacctaacggagtgctccggacctcttcagggtataagtttcctctctatatgatcggccacgggatgttggacagcgatctgcatctgtcttccaaagcccaggtgtttgaacacccacacattcaagacgccgccagccagctgccagatgatgagtcgcttttctttggtgacacaggcttgagtaagaatcccattgagctggtagaaggatggttttcatcctggaaaagttcaatagcttcctttttcttcattattggcttaattatcggcttgtttttggtcctacgtgttggtattcatctttgtatcaaactcaagcatactaagaagcgacaaatctacacagatatcgagatgaataggctgggcaagtga

SEQ ID NO: 20 - protein, 511 aa, Artificial Sequence; identical to SEQ ID NO: 17
  Synthetic VSVG AA

SEQ ID NO: 21 - DNA, 477 bp, Artificial Sequence
  Synthetic Human growth hormone polyA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 22 - DNA, 5127 bp, Artificial Sequence
  Synthetic Transfer Plasmid no Transgene
taatcccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtgcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagataagcttggtg

SEQ ID NO: 23 - DNA, 810 bp, Artificial Sequence
  Synthetic Liver codon optimized kanamycin resistance gene
tcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcat

SEQ ID NO: 24 - DNA, 41 bp, Artificial Sequence
  Synthetic HIV antisense protein start codon disruption
gcaactcacagtctggggcaacaagcagctccaggcaagaa

SEQ ID NO: 25 - DNA, 93 bp, Artificial Sequence
  Synthetic 6 frame triple stop codon insulator
tcagctacttattaattaggtgactagttgattaaaaccggtgcggccgccgccggtgtaattaggtgactagttgattaaatcagctactta

SEQ ID NO: 26 - DNA, 22 bp, Artificial Sequence
  Synthetic Transgene insertion site
accggtgcggccgccgccggtg

SEQ ID NO: 27 - DNA, 139 bp, Artificial Sequence
  Synthetic Integrase stop codon
ggcagtattcatccacaattttaaaagaaaaggggggattggggggtacagtgcaggggaaagaatagtagacataatagcaacagacatacaaactaaagaattacaaaaacaaattacaaaaattcaaaatttttaa

SEQ ID NO: 28 - DNA, 10729 bp, Artificial Sequence
  Synthetic Transfer plasmid
tccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttcagaaaaactcgtcgagcatgaggtgaaactgaagcttgttcatatctgggttatcgatgccatatttctgaaagagccgcttctgtagggaaggagagaactcaccgaggcagttccataggatggccaggtcctggtatcggtcagcgattcccactcttccaacatcaatacagcctatgagcttcccctcgtcgaaaataaggttgtccaggctgaagtccccatgagtgacgactgaatcgggactgaatggcagaagcttgtgcatttctttccagacttgctccacaggccagccattacgctcgtcgtcaaagtcggaggcgtccaccaagccgttgttcattcgactttgggcctgagcgagtctgaataccctgtcgctgttaaagggacaattgcaaacaggaatcgaatgcaaccggcgcaggaacactgccagggcatccacgatattttcaccggagtctggatactcttccagcacctggaatgctgtcttgccggggatcgcagtggtgagtaaccatgcgtcatcgggggtcctgataaagtgcttgatggtgggcagaggcatgaattccgtcagccagttcagtctcaccatctcgtcggtcacatcattggccacgcttccttttccatgtttcaggaacaactctggggcatcgggcttcccgtacaggcggtagattgtggcgcctgactgcccgacattgtcgcgagcccacttgtacccatacagatcagcatccatgttggaattcaggcgcggcctagaggtttcccgttgaatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcagccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcaacaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcagtattcatccacaattttaaaagaaaaggggggattggggggtacagtgcaggggaaagaatagtagacataatagcaacagacatacaaactaaagaattacaaaaacaaattacaaaaattcaaaatttttaatcagctacttattaattaggtgactagttgattaaaaccggtagcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagattcgagccaccatgcagctggagctctcaacctgtgtgttcctctgcctgctccccctgggattttcagctatcaggagatactatctgggagcagtggaactgtcctgggactacaggcagtcagagctgctcagagaactgcatgtggatactaggttccctgcaacagctcctggagcactgccactgggaccttcagtgctgtacaagaaaactgtctttgtggagtttacagaccagctgttcagtgtggccaggcccaggcccccctggatggggctgctgggacccaccatccaggctgaagtgtatgatactgtggtggtgaccctgaaaaacatggcctctcatccagtcagcctgcatgctgtgggagtgagcttctggaagagcagtgagggagctgagtatgaagaccatacctcacagagggagaaagaagatgataaggtgctgccaggaaaaagccagacctatgtgtggcaggtgctgaaggagaatggccctacagcttcagatcctccctgcctcacatactcttatctgagccatgtggatctggtgaaggacctcaatagtggcctgattggggcactgctggtgtgcagagaggggtccctcacaagggaaagaactcagaacctgcatgagtttgtcctgctctttgctgtgtttgatgagggaaagtcctggcactcagcaaggaatgacagctggaccagggctatggacccagcaccagccagagctcagccagctatgcacactgtcaatggctatgtgaataggtccctgcctggactcattggctgccataagaaatcagtctattggcatgtgattggaatgggcaccagcccagaggtgcattccatcttcctggaaggccacacatttctggtcaggcaccatagacaggccagcctggagatcagcccactgactttcctcacagcacagacatttctgatggacctggggcagttcctgctcttttgccatatctcaagtcaccatcatggagggatggaggctcatgtcagggtggaaagctgtgcagaggaacctcagctgaggaggaaggcagatgaggaagaggactatgatgataacctgtatgactcagatatggatgtggtgaggctggatggagatgatgtcagcccattcatccagatcaggtcagtggctaagaaacaccctaagacctgggtccactacattgcagctgaagaggaagattgggactatgcacccctggtgctggccccagatgatagaagttacaaatctcagtatctgaacaatgggccccagaggattggaaggaagtacaagaaagtgaggttcatggcttatactgatgagacctttaagacaagagaggcaatccagcatgaaagtggcatcctgggaccactgctctatggagaagtgggggataccctgctcatcatcttcaagaaccaggcctcaaggccttacaatatctatccccatggcatcacagatgtgaggcctctctacagcaggagactgcccaagggagtcaaacacctcaaggatttccccatcctgccaggggaaatcttcaagtataaatggacagtcactgtggaagatgggccaactaagtcagatcctaggtgcctgaccaggtactattctagctttgtgaacatggagagggacctggcttcaggactgattggacctctgctcatctgctacaaagaatcagtggaccagaggggcaaccagatcatgagtgataagagaaatgtcatcctgttctcagtgtttgatgagaataggagttggtatctgacagaaaacatccagaggttcctgcctaatcctgcaggagtgcagctggaggacccagaatttcaggcttcaaacatcatgcatagtatcaatggctatgtgtttgatagtctgcagctctctgtctgcctgcatgaggtggcctactggtatatcctcagcattggagctcagactgacttcctgagtgtgttcttttcaggctacacattcaagcataagatggtctatgaagataccctgacactcttccccttttctggggagactgtgtttatgagcatggaaaacccaggcctgtggattctggggtgccacaacagtgacttcaggaatagagggatgactgctctgctcaaagtgtcctcatgtgataagaatactggagattactatgaggactcttatgaagatatcagtgcatatctgctctccaaaaacaatgccattgagcccaggtcatttgctcagaacagtagaccaccttctgcaagtgcaccaaagcctccagtgctgaggagacaccagagggacatcagcctgccaaccttccagcctgaggaagataaaatggactatgatgatatcttctccactgagaccaagggggaagattttgacatctatggagaggatgaaaaccaggaccccaggtccttccagaagaggaccagacactactttattgcagctgtggagcagctgtgggactatggcatgtctgaatcacctagagctctgaggaacagagcacagaatggggaggtgcccaggttcaagaaagtggtgttcagagaatttgcagatggctcttttacccagcctagctacaggggggagctcaacaagcatctggggctgctgggaccctatatcagagcagaggtggaagataacatcatggtgacattcaagaatcaggcctcaagaccctacagtttttatagttctctgatcagctacccagatgatcaggagcagggggctgaaccaaggcacaactttgtgcagcctaatgagacaagaacttacttttggaaggtccagcatcacatggctcccacagaggatgagtttgactgcaaggcctgggcatatttttctgatgtggacctggagaaggatgtgcatagtggcctcattgggccactgctcatctgcagggcaaacacactgaatgctgcacatggcaggcaggtcactgtgcaggagtttgccctgttctttacaatctttgatgaaactaagtcctggtacttcacagagaatgtggaaaggaattgcagagccccctgccatctccagatggaggacccaactctgaaggaaaactacaggttccatgctatcaatggatatgtcatggatacactgccaggcctggtgatggcacagaaccagaggatcaggtggtatctgctcagcatggggtccaatgagaatatccattctatccacttctcaggacatgtcttttcagtgaggaagaaagaggaatataaaatggctgtgtacaatctgtatccaggggtctttgagacagtggaaatgctgcctagcaaagtggggatctggagaattgagtgcctcattggagaacacctgcaggcagggatgtccaccacatttctggtgtactcaaagaaatgccagactcccctggggatggcaagtggacatatcagggacttccagatcactgcatcaggacagtatggacagtgggcaccaaagctggctaggctccactatagtggctctatcaatgcttggagtaccaaagagcctttctcttggatcaaggtggatctgctggcccccatgatcatccatggaatcaaaacacagggagctagacagaagttcagctccctgtacatcagtcagtttatcatcatgtattctctggatgggaagaaatggcagacctacaggggcaatagcactgggacactgatggtcttctttggaaatgtggattcaagtggcatcaagcacaacatcttcaatcctcccatcattgccaggtacatcagactgcatcccacacactattcaatcaggagtactctcagaatggagctgatggggtgtgacctcaacagctgctccatgccactgggaatggaatccaaggcaatctcagatgcccagatcactgcttctagctacttcaccaatatgtttgcaacatggtcacccagtaaagcaaggctgcacctccagggaaggtccaatgcttggagaccccaggtgaacaatccaaaggagtggctgcaggtggactttcagaaaaccatgaaggtcacaggggtgactacccagggagtgaaaagtctgctcacctctatgtatgtcaaggagttcctgatctcctcaagtcaggatggccaccagtggacactgttctttcagaatggcaaggtcaaagtgttccaggggaatcaggacagctttacaccagtggtgaacagcctggacccccctctgctcactagatatctgagaatccatccacagagctgggtgcaccagattgcactcagaatggaggtcctgggctgtgaagcccaggacctgtattgagcggccccggtgaagggcgaattccagcacactggcggccgccgccggtgtaattaggtgactagttgattaaatcagctacttatccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaggggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgcctta

SEQ ID NO: 29 - DNA, 1165 bp, Artificial Sequence
  Synthetic Modified EF1a promoter
gcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 30 - DNA, 4404 bp, Artificial Sequence
  Synthetic MCO ET3, Synthetic fVIII
atgcagctggagctctcaacctgtgtgttcctctgcctgctccccctgggattttcagctatcaggagatactatctgggagcagtggaactgtcctgggactacaggcagtcagagctgctcagagaactgcatgtggatactaggttccctgcaacagctcctggagcactgccactgggaccttcagtgctgtacaagaaaactgtctttgtggagtttacagaccagctgttcagtgtggccaggcccaggcccccctggatggggctgctgggacccaccatccaggctgaagtgtatgatactgtggtggtgaccctgaaaaacatggcctctcatccagtcagcctgcatgctgtgggagtgagcttctggaagagcagtgagggagctgagtatgaagaccatacctcacagagggagaaagaagatgataaggtgctgccaggaaaaagccagacctatgtgtggcaggtgctgaaggagaatggccctacagcttcagatcctccctgcctcacatactcttatctgagccatgtggatctggtgaaggacctcaatagtggcctgattggggcactgctggtgtgcagagaggggtccctcacaagggaaagaactcagaacctgcatgagtttgtcctgctctttgctgtgtttgatgagggaaagtcctggcactcagcaaggaatgacagctggaccagggctatggacccagcaccagccagagctcagccagctatgcacactgtcaatggctatgtgaataggtccctgcctggactcattggctgccataagaaatcagtctattggcatgtgattggaatgggcaccagcccagaggtgcattccatcttcctggaaggccacacatttctggtcaggcaccatagacaggccagcctggagatcagcccactgactttcctcacagcacagacatttctgatggacctggggcagttcctgctcttttgccatatctcaagtcaccatcatggagggatggaggctcatgtcagggtggaaagctgtgcagaggaacctcagctgaggaggaaggcagatgaggaagaggactatgatgataacctgtatgactcagatatggatgtggtgaggctggatggagatgatgtcagcccattcatccagatcaggtcagtggctaagaaacaccctaagacctgggtccactacattgcagctgaagaggaagattgggactatgcacccctggtgctggccccagatgatagaagttacaaatctcagtatctgaacaatgggccccagaggattggaaggaagtacaagaaagtgaggttcatggcttatactgatgagacctttaagacaagagaggcaatccagcatgaaagtggcatcctgggaccactgctctatggagaagtgggggataccctgctcatcatcttcaagaaccaggcctcaaggccttacaatatctatccccatggcatcacagatgtgaggcctctctacagcaggagactgcccaagggagtcaaacacctcaaggatttccccatcctgccaggggaaatcttcaagtataaatggacagtcactgtggaagatgggccaactaagtcagatcctaggtgcctgaccaggtactattctagctttgtgaacatggagagggacctggcttcaggactgattggacctctgctcatctgctacaaagaatcagtggaccagaggggcaaccagatcatgagtgataagagaaatgtcatcctgttctcagtgtttgatgagaataggagttggtatctgacagaaaacatccagaggttcctgcctaatcctgcaggagtgcagctggaggacccagaatttcaggcttcaaacatcatgcatagtatcaatggctatgtgtttgatagtctgcagctctctgtctgcctgcatgaggtggcctactggtatatcctcagcattggagctcagactgacttcctgagtgtgttcttttcaggctacacattcaagcataagatggtctatgaagataccctgacactcttccccttttctggggagactgtgtttatgagcatggaaaacccaggcctgtggattctggggtgccacaacagtgacttcaggaatagagggatgactgctctgctcaaagtgtcctcatgtgataagaatactggagattactatgaggactcttatgaagatatcagtgcatatctgctctccaaaaacaatgccattgagcccaggtcatttgctcagaacagtagaccaccttctgcaagtgcaccaaagcctccagtgctgaggagacaccagagggacatcagcctgccaaccttccagcctgaggaagataaaatggactatgatgatatcttctccactgagaccaagggggaagattttgacatctatggagaggatgaaaaccaggaccccaggtccttccagaagaggaccagacactactttattgcagctgtggagcagctgtgggactatggcatgtctgaatcacctagagctctgaggaacagagcacagaatggggaggtgcccaggttcaagaaagtggtgttcagagaatttgcagatggctcttttacccagcctagctacaggggggagctcaacaagcatctggggctgctgggaccctatatcagagcagaggtggaagataacatcatggtgacattcaagaatcaggcctcaagaccctacagtttttatagttctctgatcagctacccagatgatcaggagcagggggctgaaccaaggcacaactttgtgcagcctaatgagacaagaacttacttttggaaggtccagcatcacatggctcccacagaggatgagtttgactgcaaggcctgggcatatttttctgatgtggacctggagaaggatgtgcatagtggcctcattgggccactgctcatctgcagggcaaacacactgaatgctgcacatggcaggcaggtcactgtgcaggagtttgccctgttctttacaatctttgatgaaactaagtcctggtacttcacagagaatgtggaaaggaattgcagagccccctgccatctccagatggaggacccaactctgaaggaaaactacaggttccatgctatcaatggatatgtcatggatacactgccaggcctggtgatggcacagaaccagaggatcaggtggtatctgctcagcatggggtccaatgagaatatccattctatccacttctcaggacatgtcttttcagtgaggaagaaagaggaatataaaatggctgtgtacaatctgtatccaggggtctttgagacagtggaaatgctgcctagcaaagtggggatctggagaattgagtgcctcattggagaacacctgcaggcagggatgtccaccacatttctggtgtactcaaagaaatgccagactcccctggggatggcaagtggacatatcagggacttccagatcactgcatcaggacagtatggacagtgggcaccaaagctggctaggctccactatagtggctctatcaatgcttggagtaccaaagagcctttctcttggatcaaggtggatctgctggcccccatgatcatccatggaatcaaaacacagggagctagacagaagttcagctccctgtacatcagtcagtttatcatcatgtattctctggatgggaagaaatggcagacctacaggggcaatagcactgggacactgatggtcttctttggaaatgtggattcaagtggcatcaagcacaacatcttcaatcctcccatcattgccaggtacatcagactgcatcccacacactattcaatcaggagtactctcagaatggagctgatggggtgtgacctcaacagctgctccatgccactgggaatggaatccaaggcaatctcagatgcccagatcactgcttctagctacttcaccaatatgtttgcaacatggtcacccagtaaagcaaggctgcacctccagggaaggtccaatgcttggagaccccaggtgaacaatccaaaggagtggctgcaggtggactttcagaaaaccatgaaggtcacaggggtgactacccagggagtgaaaagtctgctcacctctatgtatgtcaaggagttcctgatctcctcaagtcaggatggccaccagtggacactgttctttcagaatggcaaggtcaaagtgttccaggggaatcaggacagctttacaccagtggtgaacagcctggacccccctctgctcactagatatctgagaatccatccacagagctgggtgcaccagattgcactcagaatggaggtcctgggctgtgaagcccaggacctgtattga

SEQ ID NO: 31 - protein, 1467 aa, Artificial Sequence; identical to SEQ ID NO: 4
  Synthetic MCO ET3, Synthetic fVIII

SEQ ID NO: 32 - DNA, 3494 bp, Artificial Sequence
  Synthetic plasmid
tcagaagaactcatcgagcatcaagtgaaactgtagcttgttcatatcaggattatcaattccgtatttctgaaatagccgtttctgcagtgatggagagaactcgccgaggcagttccagaggatggcaagatcctggtatcggtcagcgattcccactcgtcctacgtcaatacagcctattaatttcccctcgtcaaaaataaggttatcaagtgagaaatcgccgtgagtcacgacactgtcaggactgaatggcaaaagtttatgcatttccttccagacttgttccacaggccagccattccgctcgtcgtcgaagtcactggcgtcaaccagaccgttattcatcctagattgtgcctgagcgagacgaaatacgcggtcgctgttaaaggggcagttacacacaggaatggaatgcaaccggcgcaggaacactgccagggcatccacaatattttcacctgaatcaggatactcttccagtacctggaatgctgtctttccggggatggcagtggtgagtaaccatgcatcatctggagtacggataaagtgcttgatggtgggcagtggcataaattctgtcagccagttgagtctgaccatctcgtcggtaacgtcattggcaacgctaccctttccatgcttcagaaacaactctggagcatcgggcttgccatacaggcgatagattgtcgcaccggactgccccacattgtccctagcccatttgtacccatacagatcagcatccatgttggaattcagtctcggcctagacgtttctctttgtatgtggctcataacaccccttgtattactgtttatgtaagcagacagttttattgttcatgatgatatatttttatcttgtgcaatgtaacatcagagattttgagacacgggccagagctgcatcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaagctagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgtaagtttagtctttttgtcttttatttcaggtcccggatccggtggtggtgcaaatcaaagaactgctcctcagtggatgttgcctttacttctagctcgagccaccatggcaggcagatcaggggactccgatgaggatctccttaaggccgtgcgtctcataaagtttctgtatcagagtaatcctccacccaaccccgaaggtacacgccaagctcggaggaatagacgtagacgctggcgagaacggcagcgacaaatccacagcatttctgagcgcattctttcgacttacttggggcggtcagccgaacctgtcccgctgcagctacccccactggagaggttgactctggattgtaacgaggactgcggtacaagtggaacccagggcgttggaagcccacagatcctggtggagtctcctaccatcttagaatccggcgctaaagagtgagcggccgcgctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaaagcttggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcct

SEQ ID NO: 33 - DNA, 810 bp, Artificial Sequence
  Synthetic kanamycin resistance gene
tcagaagaactcatcgagcatcaagtgaaactgtagcttgttcatatcaggattatcaattccgtatttctgaaatagccgtttctgcagtgatggagagaactcgccgaggcagttccagaggatggcaagatcctggtatcggtcagcgattcccactcgtcctacgtcaatacagcctattaatttcccctcgtcaaaaataaggttatcaagtgagaaatcgccgtgagtcacgacactgtcaggactgaatggcaaaagtttatgcatttccttccagacttgttccacaggccagccattccgctcgtcgtcgaagtcactggcgtcaaccagaccgttattcatcctagattgtgcctgagcgagacgaaatacgcggtcgctgttaaaggggcagttacacacaggaatggaatgcaaccggcgcaggaacactgccagggcatccacaatattttcacctgaatcaggatactcttccagtacctggaatgctgtctttccggggatggcagtggtgagtaaccatgcatcatctggagtacggataaagtgcttgatggtgggcagtggcataaattctgtcagccagttgagtctgaccatctcgtcggtaacgtcattggcaacgctaccctttccatgcttcagaaacaactctggagcatcgggcttgccatacaggcgatagattgtcgcaccggactgccccacattgtccctagcccatttgtacccatacagatcagcatccatgttggaattcagtctcggcctagacgtttctctttgtatgtggctcat

SEQ ID NO: 34 - protein, 269 aa, Artificial Sequence; identical to SEQ ID NO: 8
  Synthetic kanamycin resist AA

SEQ ID NO: 35 - DNA, 497 bp, Artificial Sequence
  Synthetic UbC promoter + SV40 intron
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttggtgtaagtttagtctttttgtcttttatttcaggtcccggatccggtggtggtgcaaatcaaagaactgctcctcagtggatgttgcctttacttctag

SEQ ID NO: 36 - DNA, 351 bp, Artificial Sequence
  Synthetic 293T codon optimized rev
atggcaggcagatcaggggactccgatgaggatctccttaaggccgtgcgtctcataaagtttctgtatcagagtaatcctccacccaaccccgaaggtacacgccaagctcggaggaatagacgtagacgctggcgagaacggcagcgacaaatccacagcatttctgagcgcattctttcgacttacttggggcggtcagccgaacctgtcccgctgcagctacccccactggagaggttgactctggattgtaacgaggactgcggtacaagtggaacccagggcgttggaagcccacagatcctggtggagtctcctaccatcttagaatccggcgctaaagagtga

SEQ ID NO: 37 - protein, 511 aa, Artificial Sequence; identical to SEQ ID NO: 17
  Synthetic 293T codon optimized rev AA

SEQ ID NO: 38 - DNA, 395 bp, Artificial Sequence
  Synthetic Beta globin polA signal
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

(19) United States
(12) Patent Application Publication
(10) Pub. No.: US 2023/0220417 A1
(43) Pub. Date: Jul. 13, 2023

(54) Lentiviral System

(71) Applicant: Expression Therapeutics, LLC, Tucker (GA)

(72) Inventor: Harrison Brown, Atlanta, GA (US)

(21) Appl. No.: 17/927,078

(22) PCT Filed: May 25, 2021
(86) PCT No.: PCT/US2021/034111

§ 371 Date: Nov. 22, 2022

Related U.S. Application Data
(60) Provisional application No. 63/029,990, filed on May 26, 2020.

(51) Int. Cl. C12N 15/86

(52) U.S. Cl. CPC C12N 15/86; C12N 2740/15043; C12N 2740/15052

(57) ABSTRACT

We disclose a lentiviral vector, for use in research, clinical, industrial, and other suitable applications. The novel lentiviral vectors disclosed herein introduce numerous novel elements which increase the safety profile of the vector without reducing the efficacy of the system. The novel lentiviral vectors are useful as safe and highly efficient transduction vectors for any application using or benefitting from transduction.

### RELATED APPLICATIONS

The present patent document claims the benefit of the filing date under 35 U.S.C. § 119(e) of Provisional U.S. Patent Application Ser. No. 63/029,990, filed May 26, 2020, which is hereby incorporated by reference in its entirety.

### REFERENCE TO APPENDIX SEQUENCE LISTING

The Sequence Listing associated with this application is provided in text format in lieu of a paper copy and is hereby incorporated by reference into the specification. The name of the text file containing the Sequence Listing is 103-3000_ST25.txt. The text file is 151,587 bytes, was created on May 21, 2021 and is being submitted electronically via EFS-Web. The information in electronic format of the Sequence Listing is incorporated herein by reference in its entirety.

### BACKGROUND

Viral vectors are a tool for delivering genetic material to target cell populations. Currently, retroviral vectors comprise approximately half of the viral gene therapy vectors in development pipelines. Of the viral vectors under development, about half are retroviral or lentiviral vectors. Retroviral vectors currently account for half of the approved gene therapy products to date.

### BRIEF SUMMARY

We disclose a viral vector, particularly a lentiviral vector, for use in research, clinical, industrial, and other suitable applications. The novel lentiviral vectors disclosed herein are useful as highly efficient transduction vectors for any application using or benefitting from transduction. Non limiting examples of potential uses of the disclosed viral vector include gene therapy, production of recombinant proteins, cancer treatment, and other manufacturing, experimental, preventative, elective, or therapeutic purposes.

### BRIEF DESCRIPTION OF THE DRAWINGS

FIG. 1 shows an exemplary HIV-1 virion.

FIG. 2 shows an exemplary retrovirus life cycle.

FIG. 3 shows basic elements of an exemplary lentiviral vector system.

FIG. 4 is a schematic of an exemplary lentiviral vector system.

FIG. 5 is a schematic of an exemplary SIN vector design.

FIG. 6 is a map of an exemplary transfer plasmid.

FIG. 7 is a map of an exemplary GAG/POL plasmid.

FIG. 8 is a map of an exemplary env(VSVG) plasmid.

FIG. 9 is a map of an exemplary transfer plasmid with a fVIII transgene.

FIG. 10 is a map of an exemplary transfer plasmid with a fVIII transgene.

FIG. 11 is a map of an exemplary REV plasmid.

FIG. 12 illustrates a potential protein product avoided by the disruption in RRE.

FIG. 13 illustrates an exemplary transgene insertion site flanked by a six frame triple stop codon (SEQ ID NO: 25).

FIG. 14 illustrates P31 integrate with terminal stop codon (SEQ ID NO: 27).

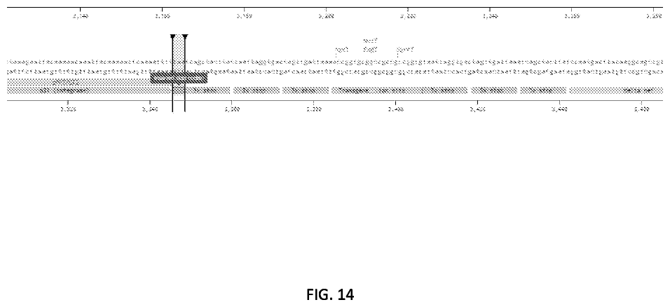

FIG. 15 illustrates an exemplary transgene design.

FIG. 16 illustrates an exemplary GAG/POL design.

FIG. 17 illustrates titers of infection titers using a disclosed lentivirus vector system.

FIG. 18 illustrates, via GFP expression, a comparison of a disclosed lentiviral vector system compared to expression in a commercially available system.

FIG. 19 illustrates, via qPCR titer, a comparison of a disclosed lentiviral vector system compared to expression in a commercially available system.

FIG. 20 illustrates, via Flow titer, a comparison of a disclosed lentiviral vector system compared to expression in a commercially available system.

FIG. 21 illustrates, via Flow titer, a comparison of earlier trial lentiviral systems where CMV promoter was used to drive expression versus two commercially available systems.

FIG. 22 illustrates, via qPCR, a comparison of earlier trial lentiviral systems where CMV promoter was used to drive expression versus two commercially available systems.

FIG. 23 illustrates, via Flow titer, a comparison of earlier trial lentiviral systems where various known promoter combinations were used to drive expression versus two commercially available systems.

### BRIEF DESCRIPTION OF THE SEQUENCES

SEQ ID NO: 1 shows the complete nucleotide sequence of a transfer plasmid of the disclosed lentiviral vector capable of expressing a proprietary liver codon optimized fVIII.

SEQ ID NO: 2 shows a nucleotide sequence of a CD68 promoter.

SEQ ID NO: 3 shows a nucleotide sequence of a liver codon optimized fVIII.

SEQ ID NO: 4 shows the amino acid sequence of a liver codon optimized fVIII.

SEQ ID NO: 5 shows the complete nucleotide sequence of a GAG/POL plasmid.

SEQ ID NO: 6 shows the nucleotide sequence of a six frame stop element.

SEQ ID NO: 7 shows the nucleotide sequence of a hCD8 codon optimized Kanamycin resistance gene.

SEQ ID NO: 8 shows the amino acid sequence of a hCD8 codon optimized Kanamycin resistance peptide.

SEQ ID NO:9 shows a nucleotide sequence of a CMV promoter and Beta globin intron.

SEQ ID NO: 10 shows a nucleotide sequence of a PGK polyA signal.

SEQ ID NO: 11 shows a nucleotide sequence of GAG.

SEQ ID NO: 12 shows an amino acid sequence of GAG.

SEQ ID NO: 13 shows a nucleotide sequence of POL.

SEQ ID NO: 14 shows an amino acid sequence of POL.

SEQ ID NO: 15 shows a nucleotide sequence of a complete ENV VSVG plasmid.

SEQ ID NO: 16 shows a nucleotide sequence of a human NK cell codon optimized Kanamycin resistance gene.

SEQ ID NO: 17 shows an amino acid sequence of a human NK cell codon optimized

Kanamycin resistance peptide.

SEQ ID NO: 18 shows a nucleotide sequence of a PGK promoter and PGK intron.

SEQ ID NO: 19 shows a nucleotide sequence for a 293T codon optimized VSVG.

SEQ ID NO: 20 shows an amino acid sequence for a VSVG.

SEQ ID NO: 21 shows a nucleotide sequence for a human growth hormone poly A.

SEQ ID NO: 22 shows a nucleotide sequence for a complete transfer plasmid with no transgene.

SEQ ID NO: 23 shows a nucleotide sequence for a liver codon optimized Kanamycin resistance gene.

SEQ ID NO: 24 shows a nucleotide sequence for a disrupted HIV antisense protein start codon.

SEQ ID NO: 25 shows a nucleotide sequence for a triple stop codon insulator sequence.

SEQ ID NO: 26 shows a nucleotide sequence for a transgene insertion site.

SEQ ID NO: 27 shows a nucleotide sequence for a P31 integrase with an added stop codon.

SEQ ID NO: 28 shows a complete nucleotide sequence of a transfer plasmid carrying the myeloid codon optimized (MCO) fVIII transgene.

SEQ ID NO: 29 shows the nucleotide sequence of a modified EF1a promoter.

SEQ ID NO: 30 shows the nucleotide sequence of a myeloid codon optimized (MCO) fVIII transgene.

SEQ ID NO: 31 shows the amino acid sequence of a MCO fVIII.

SEQ ID NO: 32 shows the nucleotide sequence of a plasmid carrying REV.

SEQ ID NO: 33 shows a nucleotide sequence of a kanamycin resistance gene.

SEQ ID NO: 34 shows an amino acid sequence of a kanamycin resistance gene.

SEQ ID NO: 35 shows a nucleotide sequence of a UbC promoter with a SV40 intron.

SEQ ID NO: 36 shows a nucleotide sequence for a 293T cell codon optimized REV gene.

SEQ ID NO: 37 shows an amino acid sequence for a 293T codon optimized REV peptide.

SEQ ID NO: 38 shows a nucleotide sequence of a Beta globin poly A signal.

### DETAILED DESCRIPTION

The use of viral vectors as a means for modification of cells, including but not limited to eukaryotic cells, is common in academia and industry for research, clinical, and manufacturing applications. Lentiviral vectors, derived from the human immunodeficiency virus, are retroviruses.

### Terms

Unless otherwise noted, technical terms are used according to conventional usage. Definitions of common terms in molecular biology may be found in Krebs et al. (eds.), Lewin's genes XII, published by Jones & Bartlett Learning, 2017; Kendrew et al. (eds.), The Encyclopedia of Molecular Biology, published by Blackwell Science Ltd., 2009 (ISBN 9780632021826). The singular terms “a,” “an,” and “the” include plural referents unless context clearly indicates otherwise. “Comprising A or B” means including A, or B, or A and B. Although methods and materials similar or equivalent to those described herein can be used in the practice or testing of the present disclosure, suitable methods and materials are described below. In addition, the materials, methods, and examples are illustrative only and not intended to be limiting. In case of conflict, the present specification, including explanations of terms, will control. In order to facilitate review of the various embodiments of the disclosure, the following explanations of specific terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Administration/Administer: To provide or give a subject an agent, such as a therapeutic agent (e.g. a recombinant AAV), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

cDNA (complementary DNA): A piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a nucleic acid sequence (including but not limited to a heterologous nucleic acid sequence) to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

Gene therapy: The introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects a function of the recipient cell.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed vectors.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as vector compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Purified: The term purified does not require absolute purity; rather, it is intended as a relative term. Thus, for example, a purified protein preparation is one in which the protein is more enriched than the peptide or protein is in its natural environment within a cell. In one embodiment, a preparation is purified such that the protein represents at least 50% of the total protein content of the preparation.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g. a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell.

Protein: A biological molecule expressed by a gene or other encoding nucleic acid (e.g., a cDNA) and comprised of amino acids.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals.

Therapeutically effective amount: The amount of agent that is sufficient to prevent, treat (including prophylaxis), reduce and/or ameliorate the symptoms and/or underlying causes of a disorder or disease.

It is understood that to obtain a therapeutic response to the disease or condition can require multiple administrations of a therapeutic agent. Thus, a therapeutically effective amount encompasses a fractional dose that contributes in combination with previous or subsequent administrations to attaining a therapeutic outcome in the patient. For example, a therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is a lentiviral vector. In some embodiments, the vector is a gamma-retroviral vector, a lentiviral vector, or an adenoviral vector.

Lentiviral Vector

Retroviruses initiate as RNA viruses that convert their RNA genome into a DNA intermediate via reverse transcriptase. The resultant DNA can then be stably integrated into the genome of the host cells in a semi-random pattern. The host cell then considers the integrated viral genome as part of its own. Therefore, the genetic regulatory elements contained within the proviral genome may affect the expression of nearby genes.

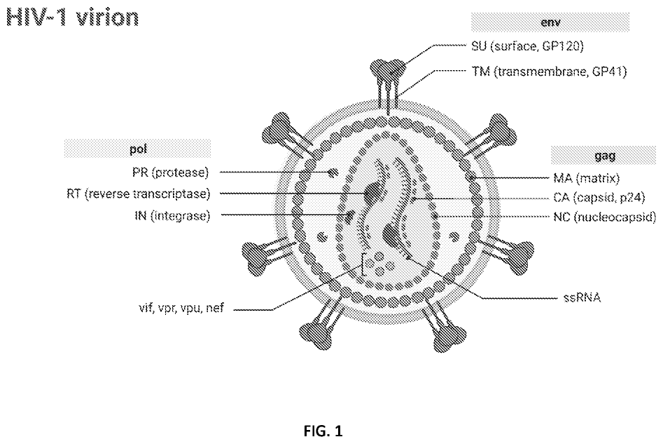

Turning to FIG. 1, using HIV-1 as a non-limiting example, we demonstrate the basic elements of the exemplary retrovirus. An outer lipid envelope may contain transmembrane and surface proteins encoded by the envelope (env) gene. The env gene may direct tropism. For example, the env gene may direct the virus to infect a particular cell, tissue, or host species. An inner proteinaceous core may include at least one of matrix, nucleocapsid and capsid proteins encoded by the gag gene, and protease, integrase and reverse transcriptase proteins encoded by the pol gene. Another genetic element found in retroviruses is the long terminal repeat (LTR) that is present on each end and possesses both promoter and enhancer activities. To summarize, basic genes useful for retroviral and lentiviral survival and function are the gag, pol, and env genes. The gag gene encodes structural proteins, pol encodes enzymes required for reverse transcriptase and integration into the host cell genome. The env gene encodes the viral envelope glycoprotein.

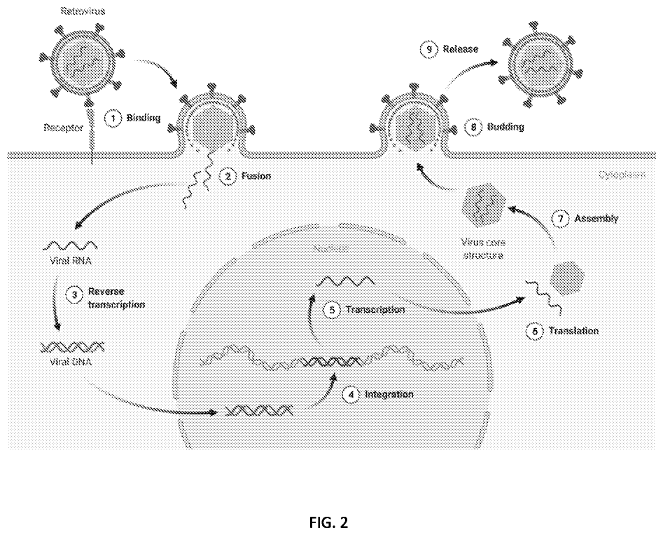

Turning to FIG. 2, the life cycle of the retrovirus includes entry by the mature virus into a cell either through membrane fusion or receptor-mediated endocytosis. In an exemplary system, after fusion, the virus proteins dissociate from the viral core. Reverse transcriptase facilitates conversion of the viral RNA into double stranded DNA. Proviral DNA complexes with the viral proteins and are transported into the host cell nucleus where it may be integrated into the host genome. Accessory viral proteins including integrase together with endogenous host cell factors assist integration of the proviral DNA into the host genome. The integrated proviral genome of the, e.g., unmodified retrovirus system relies on host machinery for transcription and translation of viral proteins necessary to assemble infectious particles or virions. The resulting virions are released into the extracellular space from the plasma membrane through a process called budding. During the budding process host cell proteins may be incorporated into the virion envelope.

Reverse transcription and integration are useful for lentiviral vector function. Following uncoating, the remaining viral nucleic acid and protein complex is often referred to as the reverse transcription complex (RTC). This RTC is actively transported to the host cell chromosomal DNA, where integration may occur. Turning to FIG. 2, steps 3 through 6, The process of reverse transcription of viral RNA to double-stranded viral DNA relies on multiple priming steps. A transfer RNA binds to the primer-binding site at the 5′ end of the viral RNA genome. The reverse transcriptase synthesizes a negative-strand of viral DNA (FIG. 2, 3). The viral RNA is degraded. The resulting single strand DNA is subsequently transferred to the 3′ end of the viral RNA to serve as a primer for the synthesis of the negative-strand viral DNA, which restores the U3RU5 sequence of the long terminal repeat (LTR). RNase H-resistant polypurine tracts primes the synthesis of the positive-strand viral DNA. Integration of the viral DNA involves the steps of tethering, 3′ processing/cleavage of a precise number of terminal nucleotides, strand transfer, and DNA repair.

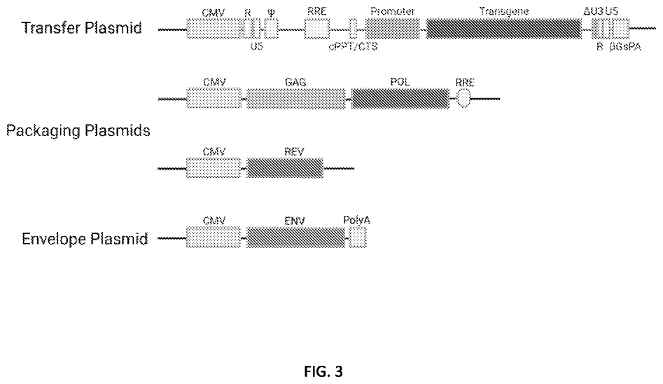

FIG. 3 illustrates the basic elements of a replication-deficient lentiviral vector system. The viral genome may be divided into separate plasmids to reduce incidence of generating recombinant virus. In this example, the viral genome is divided into three (3) separate plasmids and delivered with a separate therapeutic transgene transfer plasmid. It will be understood by one of skill in the art that the genetic elements may be further divided, combined, or reorganized onto more or fewer plasmids. In this exemplary system, the vector transfer plasmid encoding the gene of interest is operatively linked to a lentiviral LTR sequences. In this example, the vector further separates the genes encoding GAG and POL onto one plasmid separate from the individual plasmids encoding each of REV and ENV. In this example, the env gene is derived from the vesicular stomatitis virus and is referred to herein as VSVG or env(VSVG).

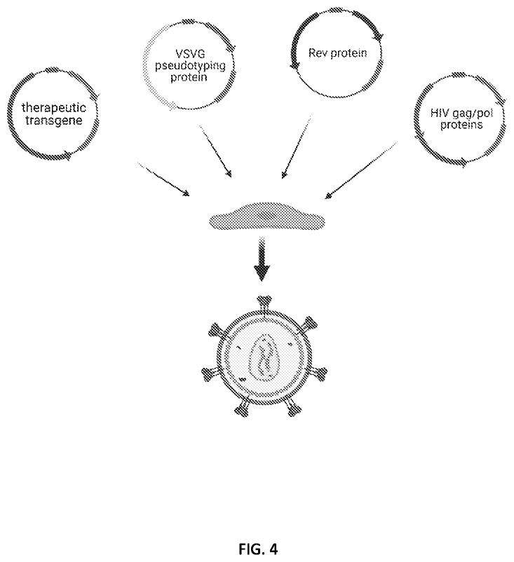

FIG. 4 is a further generic representation of the disclosed system. Theoretical safety issues that are addressed and ameliorated by the disclosed lentiviral system include but are not limited to the presence or development of replication competent virus, insertional mutagenesis, and confirmation of vector identity, purity, and manufacturing consistency. The therapeutic transgene transfer plasmid packages the desired gene that is ultimately integrated and expressed in the host cell DNA. Thus, of the various plasmids in the lentiviral vector system, the therapeutic transgene transfer plasmid has the highest safety concerns. We disclose herein various novel modifications to the lentiviral vector system to address these safety concerns.

Turning to FIG. 4, the disclosed lentiviral vector system includes a therapeutic protein transfer plasmid capable of carrying the transgene of interest; a plasmid encoding ENV, referred to herein as the VSVG pseudotyping protein; a plasmid encoding the REV protein; and a plasmid encoding the GAG and POL proteins. Other plasmids may be included and still fall within the disclosed system and/or the disclosed recombinantly modified genes may be broken into more or combined into fewer plasmids or reorganized.

Conventional lentiviral systems are nearly identical in design. To avoid copying conventional systems, the original wild type HIV genome was adopted as a starting point for modification. This removed the various single nucleotide polymorphisms (SNPs) that are ubiquitous in conventional lentiviral systems. Rather than adapting pre-existing lentiviral materials, the disclosed lentiviral system is de novo. They were custom synthesized from wild type sequence as compared to making additive, subtractive, or rearrangement changes to an existing system.

Starting with the HIV-1 HBX2 genome, we designed the disclosed novel retroviral vector. We first designed a transfer cassette which supplied the minimal cis-regulatory elements useful to support viral particle formation and proviral genome integration while eliminating or replacing genes related to viral replication This was accomplished by removing most of the viral protein encoding genetic sequence from the vector genome. A further step to eliminate viral replication was placing the required elements into separate plasmids or integrated cistrons. Here the vector components were split between 4 or more distinct elements and in some cases each element has been further modified at the nucleic acid level to reduce homology between the individual components or with natural viral sequences that could be present. (See, e.g, FIG. 4).

FIG. 3 depicts a four plasmid, lentiviral vector production system consisting of a transfer plasmid containing the LTR sequence, packaging signal, an internal promoter and the transgene of interest as well as a packaging plasmid encoding the GAG and POL proteins, another plasmid encoding REV and lastly a plasmid encoding an ENV protein. In a variation, the envelope protein may be the G protein from vesicular stomatitis virus (VSVG). The ENV protein, including but not limited to the VSVG, may confer tropism. Recent conventional packaging systems place all the vector components on individual plasmids, use inducible promoters and use codon optimization of the viral component genes.

The currently disclosed lentiviral vector system has been optimized to address drawbacks of conventional lentiviral vector systems. Among others, the system reduces the likelihood of recombination to replication-competent retrovirus (RCR), impedes mobilization of vector RNA in the case of RCR superinfection, increases the autonomy and reduces competition for transcription factors of promoters driving the transcription of the proviral RNA and accessory plasmid coding DNA sequences within cells co-transfected with the plasmid system and theoretically reduces the risk of insertional upregulation of neighboring alleles depending on the choice of the internal enhancer/promoter. The disclosed lentiviral vector also decreases the chance of expression of, e.g., out of frame polypeptides, out of context polypeptides, non-native human polypeptides, HIV polypeptides, non-native HIV polypeptides, as well as non-naturally occurring polypeptides. The disclosed lentiviral vector system achieves these features without comprising the potency or expression of the integrated transgene allele as compared to commercially available and conventional systems.

The disclosed lentiviral vector system thus decreases the time and intensiveness of regulatory evaluation. The disclosed lentiviral system also increases the efficacy of the system, e.g., when used as a gene therapy platform. Some current gene therapy interventions display poor durability, including a lack of effectiveness over time. The disclosed lentiviral vector system decreases at least one potential cause of decreased efficacy in the nature of increased immune clearance of therapeutics due to aberrant gene products, including natural or unnatural products and also including HIV proteins. Production of any aberrant polypeptides can lead to decreased efficacy.

The disclosed lentiviral system improves safety as compared to conventional lentiviral systems without substantively reducing efficacy, e.g., while maintaining approximately comparable titers with those conventional lentiviral systems.

Reduction of RCR/RCL

Currently there have been no discoverable reports of Replication competent retrovirus (RCR) or replication competent lentivirus (RCL) being detected in third or fourth generation lentiviral products. Despite this recent history of safety, evidence of RCR/RCL has been documented in preclinical studies using first generation vectors and FDA guidance still recommends RCR/RCL testing on all vector production lots, ex vivo transduced cell products and specific patient samples out to 15 years post administration. From a sponsor perspective, this is a labor and time intensive endeavor which can take as much as a month to complete.

Insertional Mutagenesis

The disclosed lentiviral system reduces incidence of insertional mutagenesis. Insertional mutagenesis is the process by which insertion of a retroviral vector into the host cell genome alters endogenous gene expression that leads to pathogenic consequences including cellular transformation and cancer development. Potential pathogenic consequences can include retroviral integration adjacent to proto-oncogenes, leading to their upregulated expression.

Vector Insertion Mediated Mutagenesis

Many types of vector insertion-mediated mutagenesis that have been described clinically which may include but are not limited to enhancer insertion, promoter insertion, insertional inactivation, and activation by 3′ end truncation.

Self Inactivating (SIN) Vector

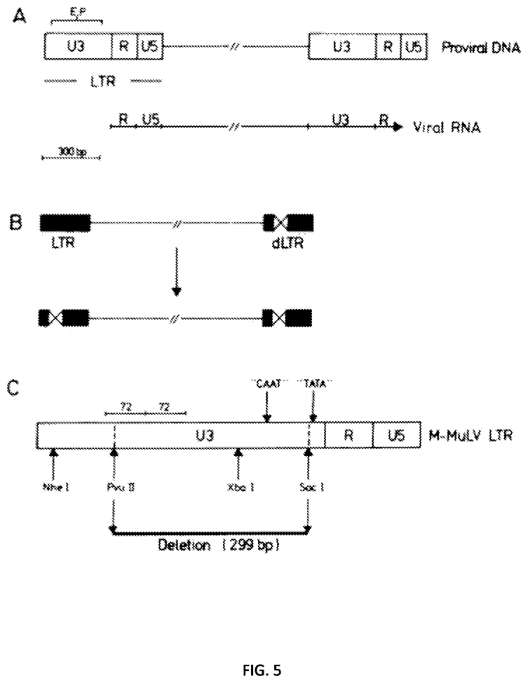

SIN vector design involves removal of critical transcriptional regulatory sequences in the U3 region of the 3′ LTR, which as shown in FIG. 5 may be used as a template to recreate the 5′ U3 sequence in the 5′ LTR of the DNA viral genome, which is lost during production of the viral RNA genome due to transcription beginning about 600 bp downstream of the beginning of the LTR. (See, e.g., S F Yu et al., Self-inactivating retroviral vectors designed for transfer of whole genes into mammalian cells. PNAS. 1986, 83 (10) 3194-3198, incorporated herein by reference in its entirety). As the U3 region is useful for the enhancer and promoter activity of retroviral LTRs, SIN vectors result in the integration of a proviral genome with both U3 regions deleted and thus no promoter/enhancer activity. In variations, an internal promoter may be utilized to drive expression of the transgene. Most known promoters are believed to have significantly less enhancer activity than the viral LTRs. Decreased enhancer activity may reduce the risk of insertional mutagenesis through enhancer activity. In a variation, additional protection may be achieved through the inclusion of insulator sequences that theoretically can further reduce the risk altering the expression of nearby genes.

Chemistry and Manufacturing Controls

Retroviral vectors are one of, if not, the most complicated biotherapeutic platform under development. Therefore, a continuing struggle has been vector manufacturing consistency and characterization during clinical development and commercialization. We disclose herein a lentivector system that implements critical advances in both the technical aspects of vector design as well as the manufacturing processes and bioanalytical methods used to characterize clinical vectors. Addressing vector identity, the disclosed lentiviral system was created through custom DNA synthesis.

Disclosed Vector

We disclose a lentiviral vector and construct which can be utilized to introduce expressible polynucleotide sequences of interest into host cells. The lentivirus vector and construct may be pseudotyped with an engineered or native viral envelope protein from another viral species, including non-lentiviruses, which may alter the host range and infectivity of lentivirus vector system. The disclosed lentiviral vectors can be used in, for example but not limited to, protein production (including vaccine production), gene therapy, delivery of therapeutic peptides, delivery of siRNA, ribozymes, ant-sense, and other functional polynucleotides, etc.

We disclose vectors comprising the nucleic acids, transcription control units, promoters and fragments thereof, optimized genes, and expression constructs disclosed herein. The vector may be of any type, for example, it may be a plasmid vector or a plasmid, baculovirus, stably expressing producer cell line, mRNA.

The efficacy of therapy is, in general, dependent upon adequate and efficient delivery of the donated transgene. This process is usually mediated by viral vectors. As such, the invention provides viral vectors, which may be based, for example, on the herpes simplex virus, adenovirus, or lentivirus. The viral vector may be a lentivirus vector or a derivative thereof, including but not limited to the HIV HXB2.

In a variation, the viral vector comprises a lentivirus vector from a naturally derived serotype or isolate, or a derivative thereof. In a further variation the disclose viral vector comprises a self-inactivating (SIN) lentivirus production system designed for delivery of therapeutic nucleic acids in, e.g., a clinical setting, manufacturing, or research setting. In a further variation the disclose viral vector comprises a self-inactivating (SIN) lentivirus production system designed with reduced homology between the plasmids that make up the system. In a further variation the reduced homology between the plasmids that make up the system is achieved by using alternative promoters, introns, and polyA combinations.

In a further variation the reduced homology between the plasmids that make up the system is achieved by using on each plasmid a unique kanamycin antibiotic resistance gene. By unique, it is meant that each kanamycin resistance gene has a different sequence from each of the other kanamycin resistance (KanR) genes. In a further variation, the kanamycin resistance genes include at least four variations in coding sequence. In a further variation, the KanR gene sequences are chosen from a liver codon optimized KanR gene, a hCD8 codon optimized KanR gene, a conventional KanR gene, and a NK Cell codon optimized KanR gene.

In a variation, we provide human CD8 cell codon optimized kanamycin resistance gene SEQ ID NO: 7. In a further variation, we provide human NK cell codon optimized kanamycin resistance gene SEQ ID NO: 16. And in a further variation, we provide liver codon optimized kanamycin resistance gene SEQ ID NO: 23.

The disclosed lentiviral vector system provides novel 293T cell codon optimized VSVG at SEQ ID NO: 19 and novel 293T cell codon optimized REV at SEQ ID NO: 36. Codon optimization increases the expression of VSVG and REV respectively when expressed in 293T cells. This is unique as most known and/or conventional systems use the standard approach of optimization for human expression using the pan-genome codon usage in the human genome. It is a unique approach to optimize for expression specifically in 293 cells using proprietary tissue specific codon optimization approach previously described (see, e.g., Brown, H. C., Zakas, P. M., George, S. N., Parker, E. T., Spencer, H. T., & Doering, C. B. (2018). Target-Cell-Directed Bioengineering Approaches for Gene Therapy of Hemophilia A. Molecular Therapy—Methods & Clinical Development, 9, 57-69. https://doi.org/10.1016/j.omtm.2018.01.004, incorporated herein by reference in its entirety.)

In a variation, multiple copies of each stop codons have been introduced to each reading frame (sense and antisense) to arrest translation of unwanted genes. Multiple stop codon strategy has been employed to at least the GAG/POL plasmid and the transfer plasmid of the disclosed lentiviral system but it should be understood that the same technique may be applied to other plasmids in the system.

In a further variation, multiple copies of each stop codons have been introduced to each reading frame (sense and antisense) to arrest translation of for example but not limited to HIV-1 vif, antisense proteins, integrase or Asp in this region before it can, e.g., result in a mutant C terminus. In a further variation, multiple copies of each stop codon have been introduced into each reading frame (sense and antisense) to reduce the chances of mutant HIV-1 vif and antisense protein expression in the GAG/POL plasmid of the system. In a further variation, a six frame stop element (SEQ ID NO: 6) has been inserted between the integrase and the RRE elements on the GAG/POL plasmid. This six frame stop element (SEQ ID NO: 6) provides insulation between the integrase and RRE elements. The six frame stop element (SEQ ID NO: 6) may be inserted on both the upstream and downstream portions. The six frame stop element also reduces the potential of translational read through between the integrase and RRE elements. By reducing the potential of translational read through the six frame stop element (SEQ ID NO: 6) reduces the potential for production of aberrant protein products. In a variation, this SEQ ID NO: 6 element provides all three stop codons in all six reading frames to reduce the potential of aberrant protein products. Potential aberrant protein products avoided may include but are not limited to NEF.

In a variation, a six frame stop element (SEQ ID NO: 25) has been inserted between on the transfer plasmid. For example, a six frame stop element (SEQ ID NO: 25) has been inserted on both the 5′ and the 3′ ends of the transgene insertion site. This six frame stop element (SEQ ID NO: 25) provides insulation between to prevent translational readthrough. By reducing the potential of translational read through the six frame stop element (SEQ ID NO: 25) reduces the potential for production of aberrant protein products. By using SEQ ID NO: 25, the region the therapeutic promoter/cDNA would be inserted into is insulated with multiple copies of each stop codon in all reading frames on both the 5′ and 3′ termini of the cloning site where the therapeutic cassette will be inserted (e.g., AgeI/SgrAI). This will again arrest any unwanted translational readthrough of either viral or therapeutic proteins in either direction. This is unique as compared to most known and/or conventional vectors which use either cloning plasmid inserts, random sequences, or multiple cutting sites in this region to provide spacing between the transgene and functional viral elements. The disclosed lentiviral system transfer plasmid used this spacer more constructively by inserting SEQ ID NO: 25 on each of the 5′ and 3′ termi of the cloning site.

In a further variation the reduced homology between the plasmids that make up the system is achieved by using on each plasmid a unique promoter. This is a diversion from the traditional practice of using the CMV promoter on each of the plasmids that make up a viral vector system. Many known recombinant lentiviral production systems use the CMV promoter to drive expression from all primary plasmids in the system, for example, if GAG/POL and REV are separated onto separate plasmids, known or conventional systems use CMV promoters to drive all four plasmids in the system. If GAG/POL and

REV are on the same plasmid, known or conventional systems use CMV promoters to drive all three plasmids in the system. By contrast, the disclosed lentivirus vector system uses a unique promoter to drive each of at least three of the four plasmids in the system. By unique, it is meant that each promoter sequence found on a plasmid has a different sequence from each of the other promoter sequences found on the other plasmids of the system.

The novel three or more unique promoter design decreases promoter competition. Promoter competition is a phenomenon by which multiple copies of the same promoter depletes the cellular supply of cytosolic transcription factors since they all have the same binding sites. This leads to decreased expression. The disclosed system includes at least one or more of the CMV promoter (SEQ ID NO: 9), the Ubiquitin C (UbC) promoter (SEQ ID NO: 35), the CD68 promoter (SEQ ID NO: 2), the PGK promoter (SEQ ID NO: 18); and the EF1a promoter (SEQ ID NO: 29). In a non-limiting variation, the REV plasmid uses Ubiquitin C promoter, the VSVG plasmid uses the PGK promoter, the GAG/POL plasmid uses the CMV promoter, the transfer plasmid uses one of the EF1a promoter, the CD68 promoter, or the CMV promoter, and the VSVG plasmid uses the PGK promoter.

In a variation, each plasmid of the system uses a distinct and unique poly adenylation signal. For example, the system includes at least one or more of the Beta globin polyA (SEQ ID NO: 38), PGK polyA (SEQ ID NO: 10), human growth hormone polyA (SEQ ID NO: 21), and the bovine growth hormone polyA. In a further variation, the REV plasmid of the disclosed lentiviral system comprises the Beta globin polyA signal; the GAG/POL plasmid comprises the PGK polyA; the transfer plasmid uses the bovine growth hormone polyA; and the VSVG plasmid uses the human growth hormone polyA. Together, the disclosed changes remove over 1,900 bases of homologous sequence from each plasmid as compared to unmodified plasmids containing comparable base genetic structure.

In a variation, the lentiviral vector provides the functional DNA elements into at least about four (4) plasmids. The at least about four plasmids include but are not limited to a transgene transfer plasmid, an ENV expression plasmid, a REV expression plasmid, and a GAG/POL expression plasmid. In a further variation, the ENV expression plasmid may be a VSVG expression plasmid.

The disclosed lentiviral vector is capable of producing synthetic lentiviral particles with an enhanced safety profile as compared to first- and second-generation lentiviral particles. Enhancing safety of the GAG/POL is not typically addressed because the GAG/POL plasmid of the lentiviral vector system is technically not transferred to a patient. Therefore, the changes and the approach of modifying the GAG/POL plasmid is a unique approach. If the lentiviral vector system is used in gene therapy applications, aberrant production of a transmembrane protein from the GAG/POL plasmid could theoretically be transferred during the budding process which would potentially deliver that protein to cells. An example of a transmembrane protein that could be transferred during the budding processing includes but is not limited to HIV antisense protein (ASP).

The disclosed lentiviral vector system may be used for any gene transfer application, including but not limited to in the clinical, manufacturing, or research setting. By reducing the homology between the plasmids that make up the lentiviral vector system, there is a reduced threat of homologous recombination between the plasmids in the lentiviral vector system. This further reduces the threat of aberrant recombination which may lead to unintentional production of replication competent virus. The disclosed lentiviral vector system produces high titer vector while incorporating novel features such as those listed above to reduce homology between plasmids and prevent translation of, e.g., unwanted protein products including but not limited to HIV protein products, native protein products, non-native natural protein products and/or non-natural protein products.

While the lentiviral system is discussed, it should be understood that the disclosed system of reducing homology between plasmids and the genes disclosed herein may be applied to the field of AAV gene therapy. The skilled person can select an appropriate serotype, clone or isolate of virus for use with the presently disclosed features including the presently disclosed gene sequences. It will be understood that this disclosure encompasses other virus and serotypes that have yet been identified or characterized.

Transfer Vector

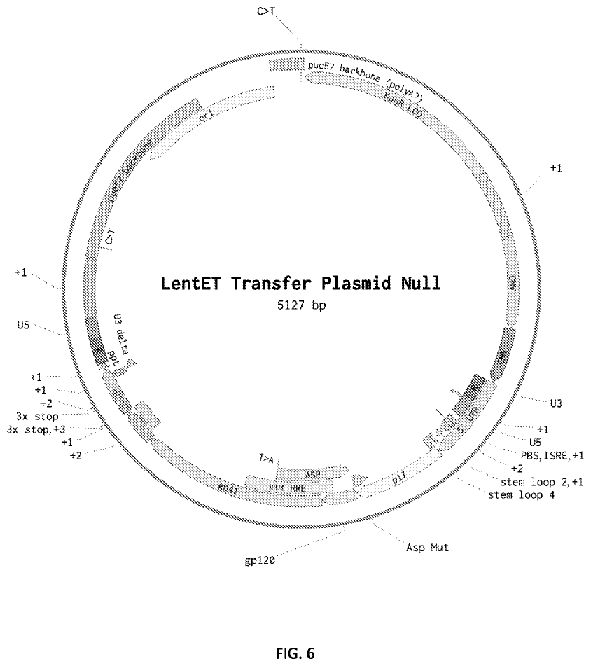

Turning to FIG. 6, we disclose a lentiviral vector system including a novel lentiviral transfer vector. A transfer vector is a construct which contains the polynucleotide sequences which are packaged into the transducing lentiviral vector.

FIG. 6 demonstrates an “empty” transfer plasmid, meaning that FIG. 6 demonstrates the basic transfer plasmid that does not include a transgene cDNA. Any desirable transgene cDNA and/or transgene and promoter can be inserted into the lentiviral vector system transfer plasmid for integration into a host cell. The system is agnostic to any particular sequence of cDNA, internal promoter, or transgene cassette

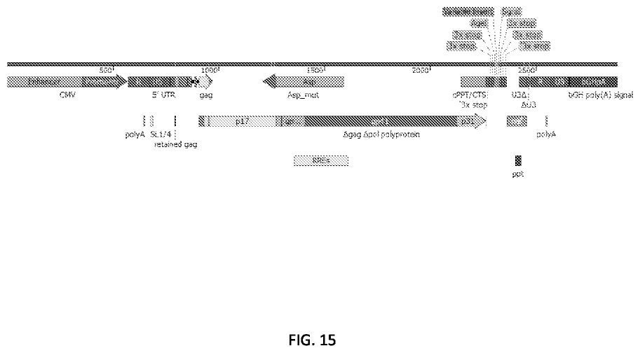

Since the basics of lentiviral vector systems are well known, we will focus our description on the novel elements of our system. For example, it should be understood that any suitable lentiviral 5′ LTR sequence, packaging sequence (psi), 3′ LTR, U3 region elements, can be placed in the transfer vector. The transfer vector can further include other additional elements, e.g., arranged in any order (with the already described elements): 5′ LTR, PBS, packaging sequence, splice donor (SD), origin of replication, optionally a central polypurine tract (PPT), RRE, MCS, splice acceptor (SA), and a modified minimally functional 3′ LTR. The expressible heterologous polynucleotide sequence can be inserted in the “variable insert” site as indicated in FIG. 15. In that variation, AgeI, SgrAI, and NotI restriction sites are present to facilitate specific, directional cloning into this site.

The transfer vector can also contain one or more SD (naturally-occurring or modified) sites. Such sequence can be intact and fully native, or it can be modified by any method known or hereafter discovered.

The disclosed lentiviral vector system transfer plasmid carries the minimal viral elements useful to permit packaging of the RNA product into lentiviral capsids. While the RNA product may be driven by any promoter, including any one of the promoters disclosed herein, FIG. 6 illustrates a transfer plasmid where the RNA product is driven by an external CMV promoter. Similarly, while the RNA product may be terminated by any polyA, including any polyA disclosed herein, FIG. 6 shows a bovine growth hormone (bGH) polyA signal.

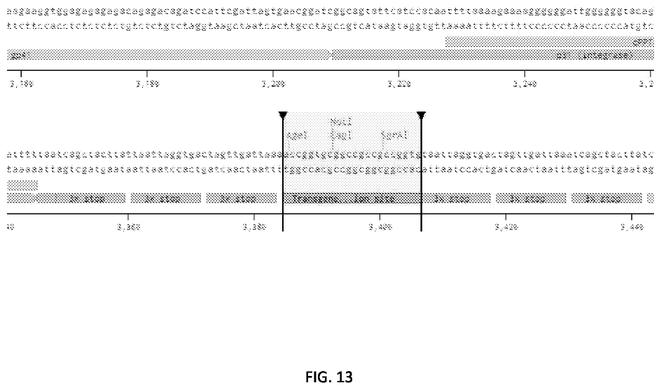

In a variation, the lentivirus vector system transfer plasmid may include at least one of a) transgene cDNA, b) one or more AgeI/NotI/SgrAI restriction sites restriction handles, c) KanR gene (which may be a codon optimized KanR gene, which may further be a liver codon optimized KanR gene), d) mutated RRE portion, e) six frame triple stop codon at one more of the 5′ and 3′ side of the transgene insertion site, f) integrase stop codon, g) promoter selected from CMV or EF1a. In a further variation, the lentivirus vector system transfer plasmid may include at least one of a) transgene cDNA, b) one or more AgeI/NotI/SgrAI restriction sites restriction handles, c) KanR gene (which may be a codon optimized KanR gene, which may further be a liver codon optimized KanR gene) (SEQ ID NO: 23), d) mutated RRE portion (SEQ ID NO: 24), specifically a mutation added at position 2672 of the RRE or its equivalent when aligned with the RRE of SEQ ID NO: 22, e) six frame triple stop codon at one more of the 5′ and 3′ side of the transgene insertion site (SEQ ID NO: 25)(See FIG. 13), f) integrase stop codon (SEQ ID NO: 27), which may be located directly after P31/integrase, g) promoter selected from CMV (SEQ ID NO: 9) or EF1a (SEQ ID NO: 29).

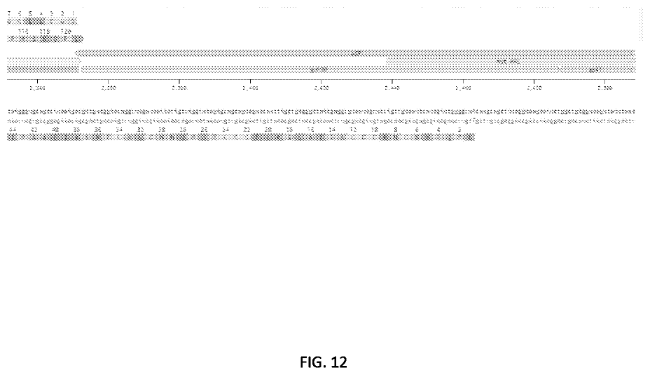

FIG. 12 illustrates the potential protein products that are avoided by the disruption introduced by SEQ ID NO: 24. In this case, a theoretical protein product (designated gp120 on FIG. 12) that is a synthetic, out of frame, unnatural protein may be avoided. FIG. 13 illustrates the P31 integrase with terminate stop codon (SEQ ID NO: 27). This was inserted to remove any possibility of read through. Although this is not predicted to be a translated region, the stop was added out of an abundance of caution. SEQ ID NO: 29 is a EF1a promoter, which contains a single base pair mutation. The single base pair mutation removes a restriction site, making cloning the transgene into the plasmid easier.

FIG. 15 provides another view of a lentivirus transgene design. In this FIG. 15, the elements have the following meaning: FIG. 15 CMV: wild type genomic human betaherpesvirus 5 isolate UCSF-1a (CMV) enhancer/promoter; Spacer: None, CMV promoter leads directly into HIV genomic sequence; 5′ UTR: 100% genomic HIV-1 genomic sequence from R to gag p17 start codon; P17: Begins at its start codon and is truncated slightly on the 3′ end where it hits gp120, Coding sequence begins with native ATG. The plasmid includes an inserted “cg” doublet which shifts reading frame and prematurely terminates p17l, the 5′ end contains final stem loop of psi region, unsure of function from the rest; gp120/41: Picks up RRE sequence as well as the HIV antisense protein (Asp), A stop codon is added to prevent translation of the full (mutant) antisense protein; gp41: Extended to be sure entire RRE is contained, functional area is not precisely defined; p31: Picks up central polypurine site; 3× stops: Spacer sequence flanking expression cassette, Provides stop codons in all 6 reading frames, cDNA is cloned in XhoI/NotI, promoters are cloned in AgeI/XhoI; 3′ UTR: U3 is deleted comparably to other designs; PolyA: Bovine growth hormone polyA (not packaged); Backbone: Puc57 kan-based, uses arbitrarily codon optimized KanR cDNAs to reduce plasmid homology.

It will be understood by those of skill in the art that the cDNA is not limited to a therapeutic product or coding DNA sequence. The disclosed lentiviral vector system may be incorporated with CRISPR, shRNA, and other therapeutic, commercial, or research strategies now known or discovered 5 after.

Accessory Constructs

We disclose a lentiviral system which includes accessory constructs (e.g., a plasmids or isolated nucleic acids). Such constructs contain the elements that are useful for producing a functional lentiviral transduction vector in a compatible host cell, and packaging into it an expressible heterologous sequence. These elements include structural proteins (e.g., the gag precursor), processing proteins (e.g., the pol precursor), such as proteases, envelope protein, and the expression and regulatory signals needed to manufacture the proteins in host cells and assemble functional viral particles. Although the embodiment described below contains the envelope and gag-pol precursor on different plasmids, they can be placed on the same plasmid, if desired, or can be further divided to include separate plasmids for each of the gag, pol, and envelope proteins.

In a variation, the lentiviral accessory plasmid can comprise one or more of the following elements in any suitable order or position, e.g., a) lentivirus 5′ LTR comprising a functional native promoter operably linked to a polynucleotide sequence coding for lentivirus gag and pol (e.g., a lentivirus gag-pol precursor); and b) heterologous promoter operably linked to an envelope coding sequence. The lentivirus 5′LTR can optionally contain heterologous enhancer sequences located upstream from the native sequence.

It will be understood by one of skill in the art that any suitable lentiviral 5′ LTR can be utilized in accordance with the present invention, including an LTR obtained from any lentivirus species, sub-species, strain or clade. This includes primate and non-primate lentiviruses.

GAG/POL

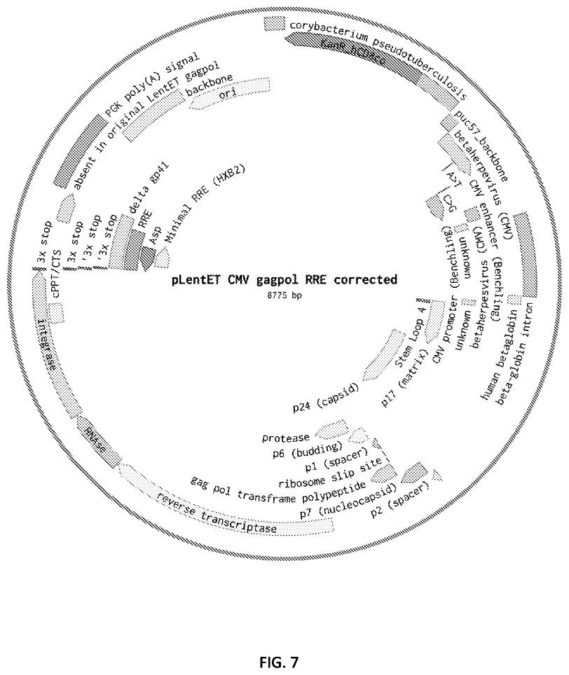

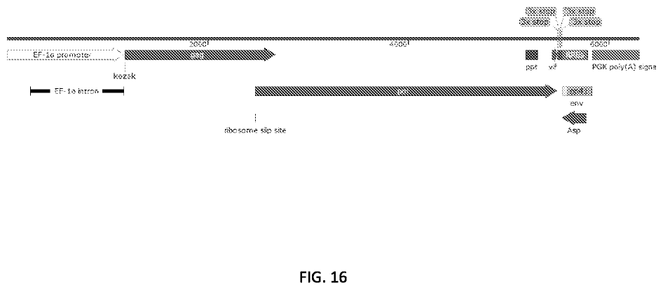

The lentiviral vector GAG/POL plasmid may provide many of the lentiviral proteins useful to produce and package lentiviral particles. In a variation, it may be drive by any promoter, including but not limited to promoters disclosed herein. FIG. 7 shows an exemplary GAG/POL plasmid of the disclosed lentiviral vector system. In that example, the GAG/POL plasmid is driven by a CMV promoter. FIG. 16 shows another view of the GAG/POL plasmid.

Native Gag-Pol sequences can be utilized in the accessory vector, or modifications can be made. Examples of possible modifications include but are not limited to, chimeric Gag-Pol, where the Gag and Pol sequences are obtained from different viruses (e.g., different species, subspecies, strains, clades, etc., and/or where the sequences have been modified to improve transcription and/or translation, and/or reduce recombination. In other embodiments of the present invention, the sequences coding for the gag and pol precursors can be separated and placed on different vector constructs, where each sequence has its own expression signals.

It will be understood by one of skill in the art that additional promoter and enhancer sequences can be placed upstream of the 5′ LTR in order to increase, improve, enhance, etc., transcription of the gag-pol precursor. Examples of useful promoters, include, mammalian promoters (e.g., constitutive, inducible, tissue-specific), CMV, RSV, LTR from other lentiviral species, and other promoters as mentioned above and below.

Safety is increased with such vectors as there is no possibility that transcriptional readthrough would result in a RNA that contains both functional gag-pol and envelope sequences. Transcriptional read through can be prevented by utilizing strong polyadenylation sequences that terminate transcription.

Turning again to FIG. 7, the encoded protein elements are p17 (HIV matrix), p24 (HIV capsid), p7 (HIV nucleocapsid), HIV P1, HIV P6, HIV protease, HIV reverse transcriptase, RNAse, and integrase also carries a rev responsive element in the 3′ UTR. It is driven by a mutated CMV promoter (SEQ ID NO: 9) which is linked to a beta-globin intron (SEQ ID NO: 9) and terminated by a PGK polyadenylation signal (SEQ ID NO: 10). Use of the PGK polyA signal (SEQ ID NO: 10) in combination with the CMV Beta-globin promoter (SEQ ID NO: 9) is a unique combination not otherwise used.

As discussed above, the GAG/POL plasmid shown on FIG. 7 includes a six frame stop element (SEQ ID NO: 6) located between the integrase and the RRE elements. It also includes a unique hCD8 codon optimized KanR gene (SEQ ID NO: 7).

Poly A

In addition, any of the plasmids in the disclosed lentivirus vector system can further comprise transcription termination signals, such as a polyA signal that is effective to terminate transcription driven by the promoter sequence. Any suitable polyA sequence can be utilized, e.g., sequences from beta globin (mammalian, human, rabbit, etc), thymidine kinase, growth hormone, SV40, and many others. The use of unique polyA signals improves safety by further reducing homology between plasmids.

ENV

The disclosed lentiviral vector construct can further comprise an accessory plasmid which is an envelope module comprising a heterologous promoter operably linked to an envelope coding sequence. The envelope polypeptide is displayed on the viral surface and is involved in the recognition and infection of host cells by a virus particle. The host range and specificity can be changed by modifying or substituting the envelope polypeptide, e.g., with an envelope expressed by a different (heterologous) viral species or which has otherwise been modified. This is called pseudotyping. See, e.g., Yee et al., Proc. Natl. Acad. Sci. USA 91: 9564-9568, 1994. Vesicular stomatitis virus (VSV) protein G (VSV G) has been used extensively because of its broad species and tissue tropism and its ability to confer physical stability and high infectivity to vector particles. See, e.g., Yee et al, Methods Cell Biol., (1994) 43:99-112

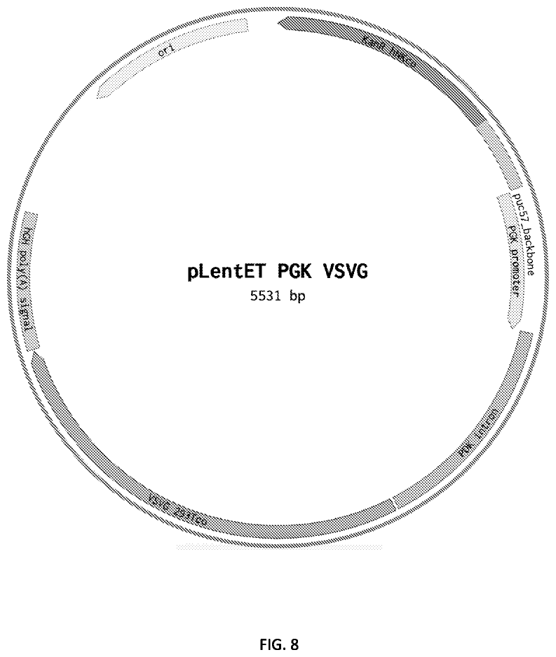

Turning to FIG. 8, we disclose a viral envelope (ENV) protein VSVG that is codon optimized for better expression in 293T producer cells. While any promoter can be used, as discussed herein, the ENV plasmid of FIG. 8 is driven by a PGK promoter linked to a PDK intron and terminated by a human growth hormone polyadenylaton signal (SED ID NO: 21). The combination of the PGK intron and the PDK promoter and the VSVG gene is unique to the disclosed lentiviral system. The PGK promoter is a unique choice to drive VSVG as compared to the most conventional choice is the CMV promoter. The CMV promoter is more powerful and is thought to be the optimal promoter to drive expression. The PGK promoter is not as strong as the CMV promoter and thus its effectiveness gave surprising results. Furthermore, the combination of the human growth hormone (hGh) polyA (SEQ ID NO: 21) with the PGK promoter and the PDK intron combination and/or with the VSVG gene is also unique to this lentiviral vector system. The VSVG coding sequence (SED ID NO: 19) was codon optimized using a unique strategy tailored specifically for expression in 293T cells. This was done using a technique we call “tissue specific codon optimization”, which seeks to mimic the codon usage bias of genes highly and specifically expressed in the target tissue/cell. (See Brown 2018.)

The lentiviral vector system ENV plasmid variation shown in FIG. 8 includes a codon optimized KanR gene that is unique from that used on other plasmids and that is codon optimized for hNK tissue specific expression (SEQ ID NO: 16). This further reduces homology between the plasmids of the lentiviral vector system.

REV

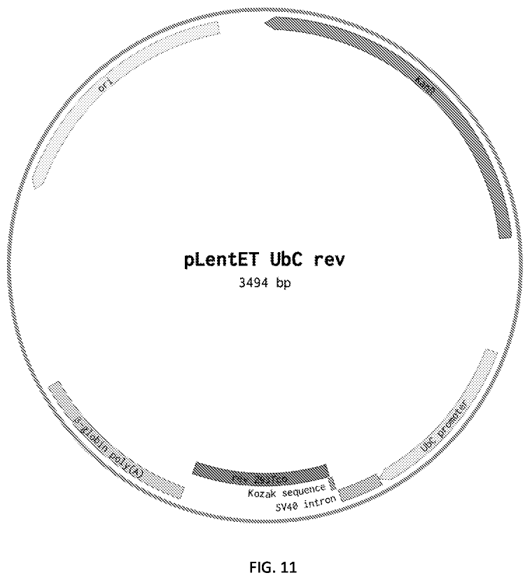

The REV plasmid encodes the HIV rev protein. REV binds the rev-responsive element and assists in nuclear export of the mRNA. Our exemplary REV plasmid is shown in FIG. 11. It is driven by a ubiquitin C (UbC) promoter linked to an SV40 intron (SEQ ID NO: 35) and terminated by a beta globin polyadenylation signal (SEQ ID NO: 38). It is unique to use the UbC promoter to drive the REV gene. The REV gene (SEQ ID NO: 36) disclosed in the REV plasmid has been codon optimized for maximum expression in HEK 293T cells.

The REV plasmid of the disclosed system includes, among other elements, a KanR gene shown in SEQ ID NO. 33, which is uniquely different in sequence from the KanR nucleotide sequences on the other plasmids in the lentiviral vector system. This further reduces the sequence homology between plasmids and increases the safety profile of the lentiviral vector as described above. The combination of the UbC promoter and the SV40 intron (SEQ ID NO: 35) with the REV gene is unique to this disclosed system. Furthermore, the combination of the beta globin polyA (SEQ ID NO: 38) with the UbC promoter and the SV40 intron and/or with the REV gene is also unique to this lentiviral vector system.

While an exemplary arrangement is provided above, it will be understood that the promoter, intron, polyA, and kanamycin genes can likely be substituted between plasmids. We have put effort into finding the optimum choice/arrangement of these elements but finding other combinations of these elements linked to coding/functional regions is within the spirit of the disclosure.

The exact interaction of the elements is very complex and still a subject of research in the HIV field. Broadly, the system functions as follows:

The 4 plasmids are co-transfected into lentivirus producer cells, often, but not exclusively, an HEK 293 cell line. After some number of hours, these plasmids are expressed in the producer cells

The VSVG plasmid drives expression of the VSVG transmembrane protein molecule. This molecule is trafficked to the surface of the producer cells.

The REV plasmid drives expression of the REV protein.

The GAG/POL plasmid drives expression of the multiple gene products coded in it. Gag and pol are produced as single polyprotein amino acid chains using an internal ribosomal slip site to change reading frames between gag and pol genes. These proteins are the structure and enzymatic proteins that allow the formation and function of the lentiviral particle.

The promoter, e.g., the CMV or EF1a promoter, driving the transfer plasmid drives expression of the RNA molecule that will be incorporated into the lentiviral molecule. This RNA includes the HIV UTRs that will assist in packaging and, in therapeutic/functional designs, an internal promoter/cDNA combination.

The REV protein product binds the REV-responsive element in the transgene RNA product and assists in nuclear export

Aided by the RRE element, which acts as a scaffold, the lentiviral molecule is formed in a manner analogous to how HIV is formed.

The forming lentiviral molecule buds off of the producer cell, where it picks up the VSVG membrane proteins present on the producer cell.

These molecules are now capable of transducing and integrating into a target cell, e.g., those expressing the LDLR.

Transgenes

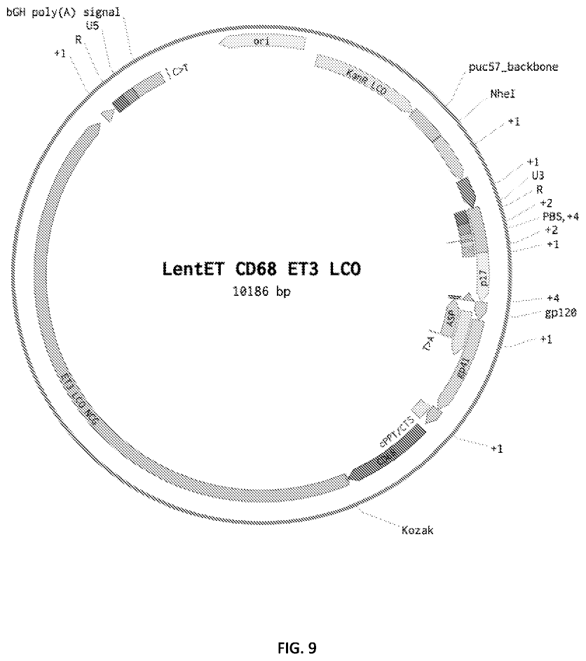

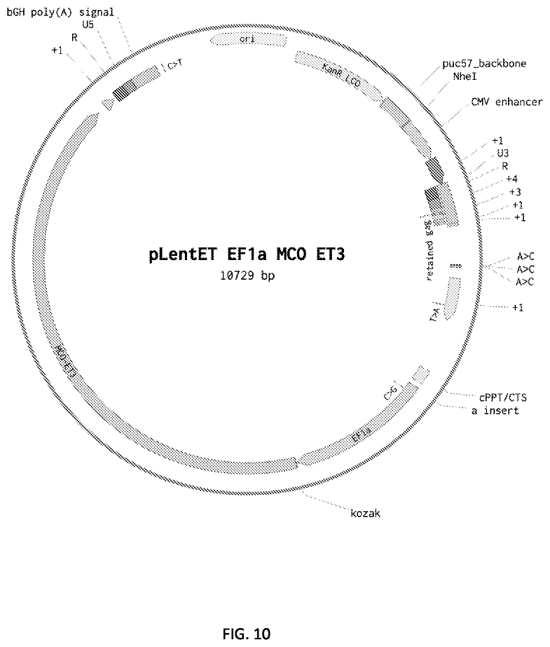

We provide sequences and vectors that demonstrate the use of various fVIII constructs in the disclosed lentivirus vector system. For example, FIG. 9 shows the disclosed lentivirus system operatively linked to a liver codon optimized fVIII gene. FIG. 10 shows the disclosed system operatively linked to a myeloid codon optimized fVIII gene. As discussed above, the lentiviral vector system is agnostic to the gene product. While fVIII is described, transgenes successfully packaged in this system include but are not limited to GFP, UNC13D, EGFP, insulin, and others.

Method of Producing

An exemplary method of producing a lentivirus vector system includes the following protocol, day by day. This is only one example and various modifications or alternative methods may be used.

The plasmids themselves may be commercially synthesized and delivered as a usable product. The are used as follows, which details how the lentivector is produced in a 6-well plate format:

293T-17 cells were grown to ˜70% confluency in tissue culture treated plates

Plasmids were transfected into cells using PEI at a ratio of 1 uL PEI to 1 ug of plasmid

Media was changed on cells 24 hours after transfection

Conditioned supernatant was collected at 48 and 72 hours post transfection

Pooled supernatant was filtered through 0.45 uM PVDF filters

Vector was titered on 293T-17 cells. Titer was determined 5 days post transduction by flow cytometry (GFP vectors) or qPCR directed against the RRE region of the lentiviral transgene cassette (GFP and FVIII vectors) against a standard curve generated from the appropriate transgene plasmid.

### EXAMPLES

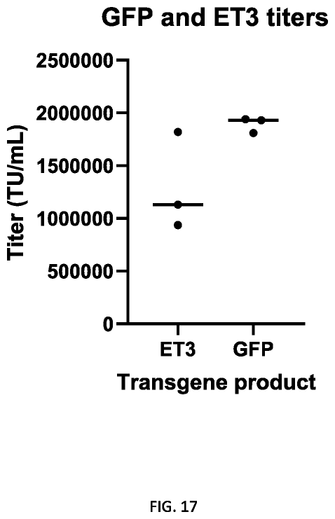

Using the above described procedure, we achieved the infection titers shown in FIG. 17 using the lentiviral vector system. These vectors were produced and titered on HEK 293T-17 cells. Titers were performed using quantitative PCR using primers directed against the RRE region of the transgene plasmid. In FIG. 17, this data shows the system produces high titer, functional vector particles. “ET3” is a human/porcine chimeric coagulation factor VIII molecule.

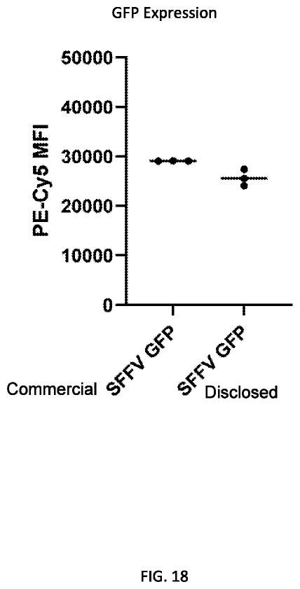

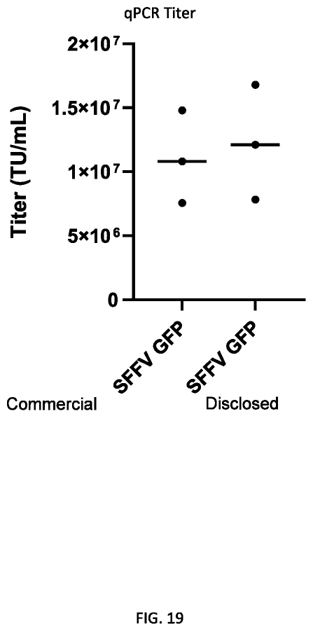

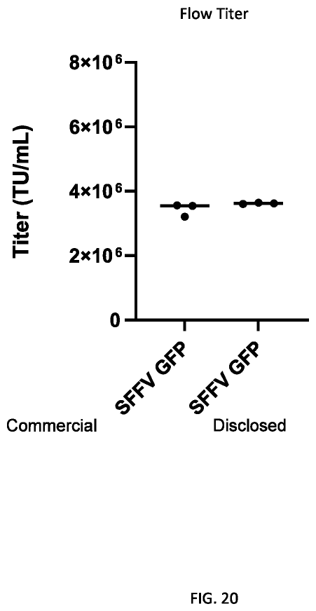

FIGS. 18 through 20 demonstrate the performance of the disclosed lentiviral vector expressing GFP as compared to an existing commercial system expressing GFP. The results show that the disclosed system with novel changes to improve the safety profile of the system can approximate the performance of commercially available systems.

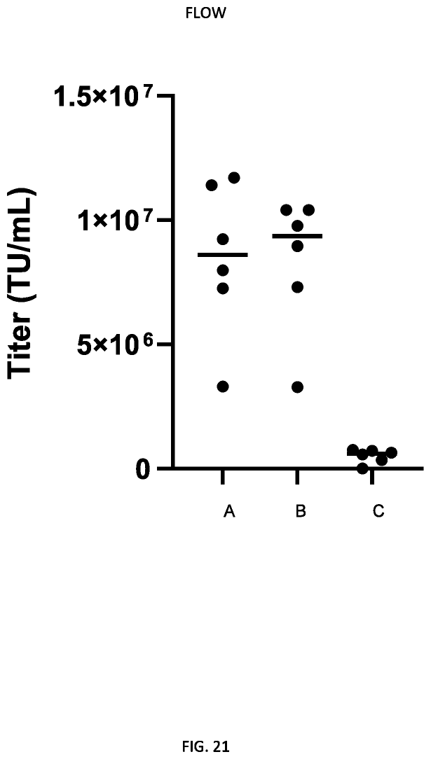

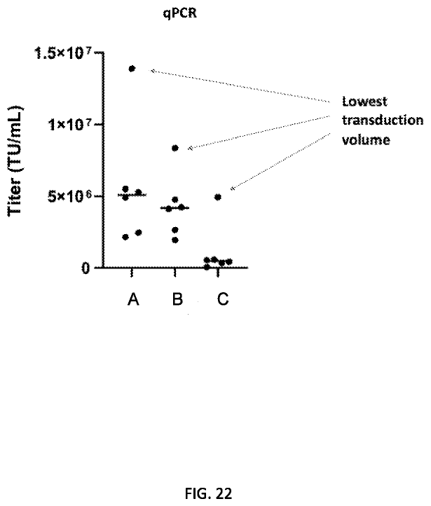

FIGS. 21 and 22 demonstrate that the optimization process met with months of lack of success before stumbling upon a combination that worked. FIG. 21 is a comparison, via Flow titer, of early lentiviral vector systems employing known techniques for vector design. In FIG. 21 we demonstrate early results of trials aimed at creating a new lentiviral system which introduced novel modifications to improve safety of the system. This evidence demonstrates that applying conventional methods resulted in poor performance relative to existing commercial systems. Because of these results, we diverged from obvious methods and explored unique methods to solve the problem. The following provides the elements used and correlates to the data in FIG. 21.

FIG. 21, A designates a first commercially available lentiviral system, FIG. 21, B designates a second commercially available system, and FIG. 21, C designates our earlier attempt systems using known vector design techniques. In FIG. 22, is a comparison, via qPCR, of early lentiviral vector systems employing known techniques for vector design compared to various commercial systems. FIG. 22, A designates a first commercially available lentiviral system, FIG. 22, B designates a second commercially available system, and FIG. 22, C designates our earlier attempt systems using known vector design techniques.

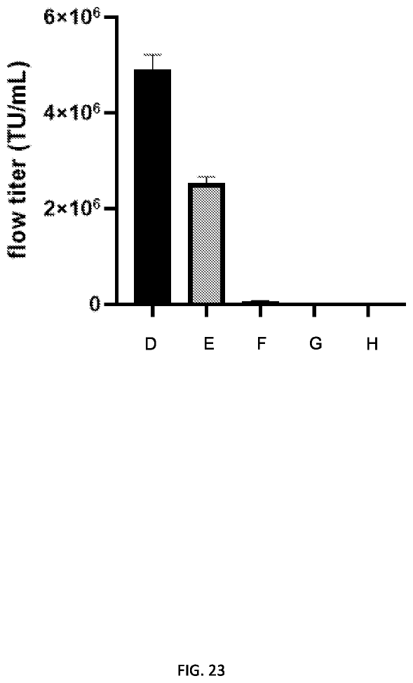

Turning to FIG. 23, the data was derived from an experiment where the transgene plasmid and the GAG/POL plasmid were maintained constant. This data demonstrates the surprising result when the CMV promoter was chosen because it is commonly used to drive expression. For example, early development employed the conventional knowledge of using the CMV promoter to drive the plasmids of vector systems. The surprising result was that the CMV promoter resulted in extremely low to no titer as shown in these figures. Only when the conventional knowledge was abandoned did we receive positive results. In FIG. 23, D is the flow titer of a first commercially available VSVG and Rev (our “goal” titer we are trying to match). We will refer to that commercially available system used as “Commercial System D.”) In FIG. 23, E is the flow titer of one of our experimental VSVG and REV (our baseline we were trying to improve on). In FIG. 23, F is the titer achieved when we adopted the CMV promoter into our early experimental system, using our VSVG with CMV and adopting REV from the Commercial System D. This severely reduced titer. In G, we used the REV from Commercial System D with our early experimental VSVG driven by the CMV promoter. This significantly decreased titer. The expected result was that substituting the CMV promoter would improve titer of the base system shown at E. In FIG. 23, H we used the CMV promoter to drive our experimental REV and VSVG, and again, the titer was decreased. The expected result was that adding CMV promoter would increase titer due to an additive effect.

For example, the CMV promoter is used to drive expression. We instead chose a promoter that is not known to have the same robust expression and yet surprisingly our titers increased to match other conventional systems while reducing overall safety profiles. FIG. 23 further demonstrates this point.

It will be apparent that the precise details of the methods or compositions described may be varied or modified without departing from the spirit of the described embodiments. For example, any of the sequences which are present in the constructs of the present invention can be modified, e.g., to improve transcription, to improve translation, to reduce or alter secondary RNA structure, and/or to decrease recombination. Modifications include, e.g., nucleotide addition, deletion, substitution, and replacements. For example, coding sequences for gag, pol, rev, and tat can be modified by replacing naturally-occurring codons with non-naturally-occurring codons, e.g., to improve translation in a host cell by substituting them with codons which are translated more effectively in the host cell. The host cell can be referred to as a compatible cell, e.g., to indicate the sequence modification has its effect when the sequence is expressed in a particular host cell type. In addition, sequences can be modified to remove regulatory elements, such as the packaging sequence. Sequences can also be altered to eliminate recombination sites. Furthermore, while the disclosed sequences are discussed in the context of the lentiviral vector system, the sequences can be used independently of the lentiviral system and/or in other systems, combinations, or arrangements.

We claim all such modifications and variations that fall within the scope and spirit of the claims below.

1. An expression vector comprising:
a. a lentiviral packaging system comprising,
i. a first plasmid comprising a structural gene selected from a GAG gene, a POL gene, or both GAG and POL genes operatively linked to a first promoter;
ii. a second plasmid comprising an ENV gene operatively linked to a second promoter;
iii. a third plasmid comprising a REV gene operatively linked to a third promoter; and
b. a lentiviral transfer plasmid; comprising a transgene operatively linked to a fourth promoter;
c. wherein at least three of the first promoter, the second promoter, the third promoter, and the fourth promoter are unique.

2. The expression vector of claim 1, the first plasmid comprising a first KanR gene, the second plasmid comprising a second KanR gene, the third plasmid comprising a third KanR gene, the fourth plasmid comprising a fourth KanR gene, and wherein at least three of the first KanR gene, the second KanR gene, the third KanR gene, and the fourth KanR gene are unique.

3. The expression vector of claim 2, where the first KanR gene is selected from a liver codon optimized KanR gene, a hCD8 codon optimized KanR gene, a conventional KanR gene, and a NK Cell codon optimized KanR gene.

4. The expression vector of claim 3, where the first KanR gene is selected from SEQ ID NO: 23, SEQ ID NO: 7, SEQ ID NO: 33, and SEQ ID NO: 16.

5. The expression vector of claim 2, where the second KanR gene is selected from a liver codon optimized KanR gene, a hCD8 codon optimized KanR gene, a conventional KanR gene, and a NK Cell codon optimized KanR gene.

6. The expression vector of claim 5, where the second KanR gene is selected from SEQ ID NO: 23, SEQ ID NO: 7, SEQ ID NO: 33, and SEQ ID NO: 16.

7. The expression vector of claim 2, where the third KanR gene is selected from a liver codon optimized KanR gene, a hCD8 codon optimized KanR gene, a conventional KanR gene, and a NK Cell codon optimized KanR gene.

8. The expression vector of claim 7, where the third KanR gene is selected from SEQ ID NO: 23, SEQ ID NO: 7, SEQ ID NO: 33, and SEQ ID NO: 16.

9. The expression vector of claim 2, where the fourth KanR gene is selected from a liver codon optimized KanR gene, a hCD8 codon optimized KanR gene, a conventional KanR gene, and a NK Cell codon optimized KanR gene.

10. The expression vector of claim 9, where the fourth KanR gene is selected from SEQ ID NO: 23, SEQ ID NO: 7, SEQ ID NO: 33, and SEQ ID NO: 16

11. The expression vector of claim 1, where the ENV gene is a 293T cell codon optimized VSVG.

12. The expression vector of claim 11, where the ENV gene is SEQ ID NO: 19.

13. The expression vector of claim 1, where the REV gene is a 293T cell codon optimized REV.

14. The expression vector of claim 13, where the REV gene is SEQ ID NO: 36.

15. A lentivirus vector system, comprising
a. A GAG/POL plasmid comprising a six frame stop element (SEQ ID NO: 6) located between an integrase element and an RRE element; a hCD8 codon optimized KanR gene;
b. A ENV plasmid comprising a hNK cell codon optimized KanR gene; and a 293T cell codon optimized VSVG gene;
c. A REV plasmid comprising a 293T cell codon optimized REV gene;
d. A transfer plasmid comprising a transgene, a liver codon optimized KanR gene, and a six frame triple stop codon of SEQ ID NO: 25 located at both the 5′ and the 3′ end of the transgene.

16. The lentivirus vector system of claim 15, the GAG/POL plasmid further comprising the hCD8 codon optimized KanR gene of SEQ ID NO: 7.

17. The lentivirus vector system of claim 15, the ENV plasmid further comprising the hNK cell codon optimized KanR gene of SEQ ID NO: 16; and the 293T cell codon optimized VSVG gene of SEQ ID NO: 19.

18. The lentivirus vector system of claim 15, the REV plasmid comprising a 293T cell codon optimized REV gene of SEQ ID NO: 36.

19. The lentivirus vector system of claim 15, the transfer plasmid further comprising the liver codon optimized KanR gene of SEQ ID NO: 23.

20. The lentivirus vector system of claim 15, the transgene encoding a fVIII polypeptide.
